(19) United States
(12) Patent Application Publication
(10) Pub. No.: US 2020/0354734 A1
(43) Pub. Date: Nov. 12, 2020

(54) NEW STRATEGIES FOR PRECISION GENOME EDITING

(71) Applicant: KWS SAAT SE & Co. KGaA, Einbeck (DE)

(72) Inventors: Markus NIESSEN, Laatzen (DE); Aaron HUMMEL, St. Louis, MO (US); Erik JONGEDIJK, Lokeren (BE); Zarir VAGHCHHIPAWALA, Ballwin, MO (US); Fridtjof WELTMEIER, Einbeck (DE); René GLENZ, Northeim (DE)

(73) Assignee: KWS SAAT SE & Co. KGaA, Einbeck (DE)

(21) Appl. No.: 16/760,100

(22) PCT Filed: Oct. 30, 2018
(86) PCT No.: PCT/EP2018/079718

§ 371 Date: Apr. 29, 2020

Related U.S. Application Data
(60) Provisional application No. 62/578,621, filed on Oct. 30, 2017.

(51) Int. Cl. C12N 15/82; C12N 15/90

(52) U.S. Cl. CPC C12N 15/8213; C12N 15/902

(57) ABSTRACT

The present invention relates to improved methods for precision genome editing (GE), preferably in eukaryotic cells, and particularly to methods for GE in cells with specifically altered expression of Polymerase theta and altered characteristics of at least one further enzyme involved in a non-homologous end-joining (NHEJ) DNA repair pathway. Further provided are cellular systems and tools related to the methods provided. Specifically, methods are provided, wherein Polymerase theta and NHEJ blockage and/or GE are performed in a transient way so that the endogenous Polymerase theta and cellular NHEJ machinery is easily reactivated after a targeted edit, and/or without permanent integration of certain editing tools.

### TECHNICAL FIELD

The present invention relates to improved methods for precision genome editing (GE), preferably in eukaryotic cells, and particularly to methods for GE in cells with specifically altered expression of Polymerase theta and altered characteristics of at least one further enzyme involved in a non-homologous end-joining (NHEJ) DNA repair pathway. The methods allow a synchronized provision of an at least partially inactivated Polymerase theta and at least one further NHEJ enzyme together with the provision of GE tools in the same cell at the time point a targeted edit is introduced to provide a significantly improved predictability and precision of the GE outcome. Further provided are cellular systems and tools related to the methods provided. Specifically, methods are provided, wherein Polymerase theta and NHEJ blockage and/or GE are performed in a transient way so that the endogenous Polymerase theta and cellular NHEJ machinery is easily reactivated after a targeted edit, and/or without permanent integration of certain editing tools.

### BACKGROUND OF THE INVENTION

The ability to precisely modify genetic material in eukaryotic cells enables a wide range of high value applications in medical, pharmaceutical, agricultural, basic research and other technical fields. Fundamentally, genome engineering or gene editing (GE) provides this capability by introducing predefined genetic variation at specific locations in eukaryotic as well as prokaryotic genomes. Recent achievements in efficient GE for targeted mutagenesis, editing, replacements, or insertions, are dependent on the ability to introduce genomic single- or double-strand breaks (DSBs) at specific locations in a genome of interest.

In eukaryotic cells, genome integrity is ensured by robust and partially redundant mechanisms for repairing DNA DSBs caused by environmental stresses and errors of cellular DNA processing machinery. In most eukaryotic cells and at most stages of the respective cell cycle, the non-homologous end-joining (NHEJ) DNA repair pathway is the highly dominant form of repair. A second pathway uses homologous recombination (HR) of similar DNA sequences to repair DSBs. This pathway can usually be used in the S and G2 stages of the cell cycle by templating from the duplicated homologous region of a paired chromosome to precisely repair the DSB. However, an artificially-provided repair template (RT) with homology to the target can also be used to repair the DSB, in a process known as homology-directed repair (HDR) or gene targeting. By this strategy it is possible to introduce very precise, targeted changes in the genomes of eukaryotic cells.

Early gene targeting studies in plants revealed frequencies of homologous recombination that were so low it was effectively impossible to practice gene editing for crop improvement. Site-specific nucleases (SSNs), which can be directed to a specific target sequence and there cause a DSB, increase gene targeting frequencies by 2-3 orders of magnitude when co-delivered together with a DNA RT (Puchta et al., Proc. Natl. Acad. Sci. USA 93:5055-5060, 1996). However, GE in plants is still hindered by low frequency of HDR repairs compared to repairs by NHEJ which can create insertions or deletions (INDELs) in the SSN target, thereby disrupting further cutting and rendering the target in a particular cell unusable for gene targeting.

An aspect to be critically considered for GE is thus the nature of the repair mechanism induced after the cleavage of a genomic target site of interest, as DSBs, or any DNA lesions in general are detrimental for the integrity of a genome. It is thus of outstanding importance that the cellular machinery provides mechanisms of double-strand break (DSB) repair in the natural environment. Cells possess intrinsic mechanisms to attempt to repair any double- or single-stranded DNA damage. DSB repair mechanisms have been divided into two major basic types, NHEJ and HR in general are usually called HDR.

NHEJ is the dominant nuclear response in animals and plants which does not require homologous sequences, but is often error-prone and thus potentially mutagenic (Wyman C., Kanaar R. “DNA double-strand break repair: all's well that ends well”, Annu. Rev. Genet., 2006, 40, 363-83). Classical- and backup-NHEJ pathways are known relying on different mechanism, wherein both pathways are error-prone. Repair by HDR requires homology, but those HDR pathways that use an intact chromosome to repair the broken one, i.e. double-strand break repair and synthesis-dependent strand annealing, are highly accurate. In the classical DSB repair pathway, the 3′ ends invade an intact homologous template then serve as a primer for DNA repair synthesis, ultimately leading to the formation of double Holliday junctions (dHJs). dHJs are four-stranded branched structures that form when elongation of the invasive strand “captures” and synthesizes DNA from the second DSB end. The individual HJs are resolved via cleavage in one of two ways. Synthesis-dependent strand annealing is conservative, and results exclusively in non-crossover events. This means that all newly synthesized sequences are present on the same molecule. Unlike the NHEJ repair pathway, following strand invasion and D loop formation in synthesis-dependent strand annealing, the newly synthesized portion of the invasive strand is displaced from the template and returned to the processed end of the non-invading strand at the other DSB end. The 3′ end of the non-invasive strand is elongated and ligated to fill the gap. There is a further pathway of HDR, called break-induced repair pathway not yet fully characterized. A central feature of this pathway is the presence of only one invasive end at a DSB that can be used for repair.

The naturally occurring NHEJ pathway, therefore, is highly efficient and a straightforward as it can assist in rejoining the two ends after a DSB independently of significant homology, whereas this efficiency is accompanied by the drawback that this process is error-prone and can be associated with insertions or deletions. The ubiquitously present NHEJ pathway in eukaryotic cells thus hampers targeted GE approaches.

A further challenge is the propensity for introduced RTs to integrate randomly into the genome at unpredictable and uncontrollable locations. One NHEJ pathway is mediated by Polymerase θ (Polymerase theta, Pol θ, or Pol theta), encoded by the POLQ gene (e.g., for plants see: van Kregten et al., 2016, T-DNA integration in plants results from polymerase-θ-mediated DNA repair. Nature Plants 2, Article number: 16164). Polymerase θ in mammals is an atypical A-family type polymerase with an N-terminal helicase-like domain, a large central domain harboring a Rad51 interaction motif, and a C-terminal polymerase domain capable of extending DNA strands from mismatched or even unmatched termini. DNA molecules can be randomly incorporated into eukaryotic genomes through the action of Pol θ being a low fidelity polymerase (Hogg et al., 2012. Promiscuous DNA synthesis by human DNA polymerase θ. Nucleic Acids Research, Volume 40, Issue 6, 1 Mar. 2012, Pages 2611-2622) that is required for random integration of T-DNAs in plants. Knockout mutant plants lacking Pol θ activity are incapable of integrating T-DNA molecules during Agrobacterium tumefaciens mediated plant transformation (van Kregten et al., 2016, supra). In vitro experiments identified an evolutionarily conserved loop in the polymerase domain that is essential for synapsing DNA ends during end joining protecting the genome against gross chromosomal rearrangements (Sfeir, The FASEB Journal, vol. 30, no. 1, 2016).

WO 2017/062754 A1 discloses GE methods in mammalian cells, focusing on mouse embryonic stem cells, wherein Pol theta is inhibited. Still, there remains the problem that the Pol theta mediated NHEJ pathway is only one of the cellular NHEJ pathways so that inhibition is not perfect and other error-prone repair pathways can hamper a targeted GE in said cell type. Furthermore, no approach is provided allowing the applicability of the disclosed methods in plant cells showing highly distinct repair mechanisms. In particular, the plant enzymes involved in error-prone repair pathways are poorly characterized making targeted GE in plant cells hard to predict. Targeted GE in plants, in particular the HDR, suffers from very low efficiency and in most crop species the delivery of the GE machinery to cells which subsequently regenerate into a transformed plant is not straightforward (e.g. protoplasts which are easy to transform do not regenerate in most crop species). Finally, there are only a few reliable methods available allowing for the isolation of the transformed cells from the majority of the untransformed cells in the tissue. These are only some difficulties the skilled artisan has to face when seeking a way to provide means for targeted GE in plant cells.

In practice, frequent random integrations of RTs limit the availability of the templates for use by cells in gene targeting, and make it difficult to screen cells or plants with the desired gene targeting events from a background of more abundant random integration events.

Thus, efficient gene targeting in eukaryotic cells is significantly hindered by low frequencies due to the prevalence of NHEJ-mediated DSB repair, and by the difficulty of screening for gene targeting events due to frequent random integration of the RT in many treated cells.

EP 2 958 996 A1 seeks to overcome the problem of specific DSB repair by providing an inhibitor of NHEJ mechanisms in cells to increase gene disruption mediated by a nuclease (e.g., ZFN or TALEN) or nuclease system (e.g., CRISPR/Cas, Cpf1, CasX or CasY). By inhibiting the critical enzymatic activities of these NHEJ DNA repair pathways, using small molecule inhibitors of DNA-dependent-protein kinase catalytic subunit (DNA-PKcs) and/or Poly-(ADP-ribose) polymerase ½ (PARP½), the level of gene disruption by nucleases is increased by forcing cells to resort to more error prone repair pathways than classic NHEJ, such as alternate NHEJ and/or microhomology mediated end-joining. Therefore, an additional chemical is added in the course of genome editing, which might, however, be disadvantageous for several cell types and assays. This could also affect the genome integrity of the treated cells and/or the regenerative potential.

Therefore, there exists an ongoing need in providing suitable strategies for precision GE in eukaryotic cells and organisms, which are also applicable in plants, especially major crop plants, which combine high precision genome cleavage and simultaneously providing the possibility for mediating highly precise and accurate HDR and thus targeted repair of a DSB, which is imperative to control a gene editing or genome engineering intervention.

It was thus an aim of the present invention to increase the predictability of GE approaches, in particular approaches applicable for plants and plant cells, wherein the outcome of a GE planned in silico can be defined in a much more accurate way by suppressing relevant NHEJ pathways in a concerted manner whilst additionally providing suitable repair templates to obtain a modified genetic material, preferably by using transient introduction strategies. Therefore, it was an objective of the present invention to unify down-regulation or knock-down of relevant NHEJ pathways with targeted GE strategies just within one cell or cellular system simultaneously to be able to introduce site-specific edits or modifications in a highly precise manner without inserting unwanted mutations or edits into a genome of interest as random—and thus not predictable—integrations during repair of a DSB artificially induced.

### SUMMARY OF THE INVENTION

The above objects have been solved by providing, in a first aspect, a method for modifying the genetic material of a cellular system at a predetermined location with at least one nucleic acid sequence of interest, wherein the method comprises the following steps: (a) providing a cellular system comprising a Polymerase theta enzyme, or a sequence encoding the same, and one or more further enzyme(s) of a NHEJ pathway, or the sequence(s) encoding the same; (b) inactivating or partially inactivating the Polymerase theta enzyme, or the sequence encoding the same, and inactivating or partially inactivating the one or more further DNA repair enzyme(s) of a NHEJ pathway, or the sequence(s) encoding the same; (c) introducing into the cellular system or a progeny system thereof (i) the at least one nucleic acid sequence of interest, optionally flanked by one or more homology sequence(s) complementary to one or more nucleic acid sequence(s) adjacent to the predetermined location, and (ii) at least one site-specific nuclease, or a sequence encoding the same, the site-specific nuclease inducing a double-strand break at the predetermined location; and (d) optionally: determining the presence of the modification at the predetermined location in the genetic material of the cellular system; (e) obtaining a cellular system comprising a modification at the predetermined location of the genetic material of the cellular system or selecting a cellular system comprising a modification at the predetermined location of the genetic material of the cellular system based on the determination of (d).

In one embodiment according to the various aspects of the present invention, there is provided a method comprising an additional step of: (f) restoring the activity of the inactivated or partially inactivated Polymerase theta enzyme and/or restoring the activity of the one or more further inactivated or partially inactivated DNA repair enzyme(s) of a NHEJ pathway in the cellular system comprising a modification at the predetermined location, or in a progeny system thereof.

In another embodiment according to the various aspects of the present invention, there is provided a method, wherein the Polymerase theta to be inactivated or partially inactivated (i) comprises an amino acid sequence according to SEQ ID NO: 2, 7, 8, 9 or 10, or (ii) comprises an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 2, 7, 8, 9 or 10, respectively, preferably over the entire length of the sequence; or (iii) is encoded by a nucleic acid sequence according to SEQ ID NO: 1, 3, 4, 5 or 6, or (iv) is encoded by a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 1, 3, 4, 5 or 6, respectively, preferably over the entire length of the sequence; or (v) is encoded by a nucleic acid sequence hybrizing to a nucleic acid sequence complementary to the nucleic acid sequence of (iii), preferably under stringent conditions.

In yet a further embodiment according to the various aspects of the present invention, there is provided a method, wherein the one or more further DNA repair enzyme(s) of a NHEJ pathway to be inactivated or partially inactivated is independently selected from the group consisting of Ku70, Ku80, DNA-dependent protein kinase, Ataxia telangiectasia mutated (ATM), ATM—and Rad3—related (ATR), Artemis, XRCC4, DNA ligase IV and XLF, or any combination thereof.

In one embodiment according to the various aspects of the present invention, at least one, at least two, at least three, or at least four further DNA repair enzymes of a NHEJ pathway are inactivated or partially inactivated, preferably wherein at least Ku70 and DNA ligase IV, or wherein at least Ku80 and DNA ligase IV are inactivated or partially inactivated.

In another embodiment according to the various aspects of the present invention, one, two, three, or four further DNA repair enzymes of a NHEJ pathway are inactivated or partially inactivated, preferably wherein Ku70 and DNA ligase IV, or wherein Ku80 and DNA ligase IV are inactivated or partially inactivated.

In one embodiment according to the various aspects of the present invention, there is provided a method, wherein the one or more further DNA repair enzyme(s) of a NHEJ pathway to be inactivated or partially inactivated is Ku70, or a nucleic acid sequence encoding the same, wherein the Ku70 comprises an amino acid sequence according to SEQ ID NO: 12, 18, 19 or 20, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 12, 18, 19 or 20, respectively, preferably over the entire length of the sequence, or wherein the nucleic acid sequence encoding the same comprises a sequence according to SEQ ID NO: 11, 13, 14, 15, 16 or 17, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 11, 13, 14, 15, 16 or 17, respectively, preferably over the entire length of the sequence, or the nucleic acid sequence hybridizes to a nucleic acid sequence complementary to the nucleic acid sequence according to SEQ ID NO: 11, 13, 14, 15, 16 or 17, preferably under stringent conditions.

In a further embodiment according to the various aspects of the present invention, there is provided a method, wherein the one or more further DNA repair enzyme(s) of a NHEJ pathway to be inactivated or partially inactivated is Ku80, or a nucleic acid sequence encoding the same, wherein the Ku80 comprises an amino acid sequence according to SEQ ID NO: 22, 23, 24 or 29, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 22, 23, 24 or 29, respectively, preferably over the entire length of the sequence, or wherein the nucleic acid sequence encoding the same comprises a sequence according to SEQ ID NO: 21, 25, 26, 27 or 28, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 21, 25, 26, 27 or 28, respectively, preferably over the entire length of the sequence, or the nucleic acid sequence hybridizes to a nucleic acid sequence complementary to the nucleic acid sequence according to SEQ ID NO: 21, 25, 26, 27 or 28, preferably under stringent conditions.

In an additional embodiment according to the various aspects of the present invention, there is provided a method, wherein the one or more further DNA repair enzyme(s) of a NHEJ pathway to be inactivated or partially inactivated is a DNA-dependent protein kinase, or a nucleic acid sequence encoding the same, wherein the DNA-dependent protein kinase comprises an amino acid sequence according to SEQ ID NO: 32, 33 or 35, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 32, 33 or 35, respectively, preferably over the entire length of the sequence, or wherein the nucleic acid sequence encoding the same comprises a sequence according to SEQ ID NO: 30, 31 or 34, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 30, 31 or 34, respectively, preferably over the entire length of the sequence, or the nucleic acid sequence hybridizes to a nucleic acid sequence complementary to the nucleic acid sequence according to SEQ ID NO: 30, 31 or 34, preferably under stringent conditions.

In a further embodiment according to the various aspects of the present invention, there is provided a method, wherein the one or more further DNA repair enzyme(s) of a NHEJ pathway to be inactivated or partially inactivated is ATM, or a nucleic acid sequence encoding the same, wherein the ATM comprises an amino acid sequence according to SEQ ID NO: 37, 38, 39, 41, 42, 43, 44, 45, 46, 47 or 48, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 37, 38, 39, 41, 42, 43, 44, 45, 46, 47 or 48, respectively, preferably over the entire length of the sequence, or wherein the nucleic acid sequence encoding the same comprises a sequence according to SEQ ID NO: 36 or 40, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 36 or 40, respectively, preferably over the entire length of the sequence, or the nucleic acid sequence hybridizes to a nucleic acid sequence complementary to the nucleic acid sequence according to SEQ ID NO: 36 or 40, preferably under stringent conditions.

In an additional embodiment according to the various aspects of the present invention, there is provided a method, wherein the one or more further DNA repair enzyme(s) of a NHEJ pathway to be inactivated or partially inactivated is ATM—and Rad3—related (ATR), or a nucleic acid sequence encoding the same, wherein the ATR comprises an amino acid sequence according to SEQ ID NO: 50, 51, 52, 53, 55 or 56, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 50, 51, 52, 53, 55 or 56, respectively, preferably over the entire length of the sequence, or wherein the nucleic acid sequence encoding the same comprises a sequence according to SEQ ID NO: 49 or 54, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 49 or 54, respectively, preferably over the entire length of the sequence, or the nucleic acid sequence hybridizes to a nucleic acid sequence complementary to the nucleic acid sequence according to SEQ ID NO: 49 or 54, preferably under stringent conditions.

In a further embodiment according to the various aspects of the present invention, there is provided a method, wherein the one or more further DNA repair enzyme(s) of a NHEJ pathway to be inactivated or partially inactivated is Artemis, or a nucleic acid sequence encoding the same, wherein the Artemis comprises an amino acid sequence according to SEQ ID NO: 60, 61, 62 or 64, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 60, 61, 62 or 64, respectively, preferably over the entire length of the sequence, or wherein the nucleic acid sequence encoding the same comprises a sequence according to SEQ ID NO: 57, 58, 59 or 63, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 57, 58, 59 or 63, respectively, preferably over the entire length of the sequence, or the nucleic acid sequence hybridizes to a nucleic acid sequence complementary to the nucleic acid sequence according to SEQ ID NO: 57, 58, 59 or 63, preferably under stringent conditions.

In an additional embodiment according to the various aspects of the present invention, there is provided a method, wherein the one or more further DNA repair enzyme(s) of a NHEJ pathway to be inactivated or partially inactivated is XRCC4, or a nucleic acid sequence encoding the same, wherein the XRCC4 comprises an amino acid sequence according to SEQ ID NO: 66, 67 or 69, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 66, 67 or 69, respectively, preferably over the entire length of the sequence, or wherein the nucleic acid sequence encoding the same comprises a sequence according to SEQ ID NO: 65 or 68, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 65 or 68, respectively, preferably over the entire length of the sequence, or the nucleic acid sequence hybridizes to a nucleic acid sequence complementary to the nucleic acid sequence according to SEQ ID NO: 65 or 68, preferably under stringent conditions.

In a further embodiment according to the various aspects of the present invention, there is provided a method, wherein the one or more further DNA repair enzyme(s) of a NHEJ pathway to be inactivated or partially inactivated is DNA ligase IV, or a nucleic acid sequence encoding the same, wherein the DNA ligase IV comprises an amino acid sequence according to SEQ ID NO: 71, 72, 76 or 77, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 71, 72, 76 or 77, respectively, preferably over the entire length of the sequence, or wherein the nucleic acid sequence encoding the same comprises a sequence according to SEQ ID NO: 70, 73, 74 or 75, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 70, 73, 74 or 75, respectively, preferably over the entire length of the sequence, or the nucleic acid sequence hybridizes to a nucleic acid sequence complementary to the nucleic acid sequence according to SEQ ID NO: 70, 73, 74 or 75, preferably under stringent conditions.

In an additional embodiment according to the various aspects of the present invention, there is provided a method, wherein the one or more further DNA repair enzyme(s) of a NHEJ pathway to be inactivated or partially inactivated is XLF, or a nucleic acid sequence encoding the same.

In another embodiment according to the various aspects of the present invention, there is provided a method, wherein the one or more further DNA repair enzyme(s) of a NHEJ pathway to be inactivated or partially inactivated are the Ku70 or the nucleic acid sequence encoding the same, and/or the Ku80 or the nucleic acid sequence encoding the same, and/or the DNA-dependent protein kinase, or the nucleic acid sequence encoding the same, and/or the ATM or the nucleic acid sequence encoding the same, and/or the ATM—and Rad3—related (ATR), or the nucleic acid sequence encoding the same, and/or the Artemis, or the nucleic acid sequence encoding the same, and/or the XRCC4, or the nucleic acid sequence encoding the same, and/or the DNA ligase IV, or the nucleic acid sequence encoding the same, and/or the XLF, or the nucleic acid sequence encoding the same.

In one embodiment according to the various aspects of the present invention, there is provided a method, wherein the at least one nucleic acid sequence of interest is provided as part of at least one genetic construct, or as at least one linear molecule.

In another embodiment according to the various aspects of the present invention, there is provided a method, wherein the at least one genetic construct is introduced into the cellular system by biological or physical means, including transfection, transformation, including transformation by Agrobacterium spp., preferably by Agrobacterium tumefaciens, a viral vector, biolistic bombardment, transfection using chemical agents, including polyethylene glycol transfection, electroporation, electro cell fusion, or any combination thereof.

In still another embodiment according to the various aspects of the present invention, there is provided a method, wherein the at least one site-specific nuclease or a part thereof, or the sequence encoding the same, is introduced into the cellular system by biological or physical means, including transfection, transformation, including transformation by Agrobacterium spp., preferably by Agrobacterium tumefaciens, a viral vector, bombardment, transfection using chemical agents, including polyethylene glycol transfection, electroporation, electro cell fusion, or any combination thereof.

Further provided is a method according to the various aspects disclosed herein, wherein the at least one site-specific nuclease or a catalytically active fragment thereof, is introduced into the cellular system as a nucleic acid sequence encoding the site-specific nuclease or the catalytically active fragment thereof, wherein the nucleic acid sequence is part of at least one genetic construct, or wherein the at least one site-specific nuclease or the catalytically active fragment thereof, is introduced into the cellular system as at least one mRNA molecule or as at least one amino acid sequence.

In one embodiment according to the various aspects of the present invention, there is provided a method, wherein the at least one nucleic acid sequence of interest to be introduced into a cellular system is selected from the group consisting of: a transgene, a cisgene, a modified endogenous gene, a codon optimized gene, a synthetic sequence, an intronic sequence, a coding sequence, or a regulatory sequence or a part thereof including a core promoter, a cis-acting element, conserved motif like TATA box et cetera.

In another embodiment according to the various aspects of the present invention, there is provided a method, wherein the at least one nucleic acid sequence of interest to be introduced into a cellular system is a transgene or cisgene, wherein the transgene or cisgene comprises a nucleic acid sequence encoding a gene of a genome of an organism of interest, or at least a part of said gene.

In still another embodiment according to the various aspects of the present invention, there is provided a method, wherein the at least one nucleic acid sequence of interest to be introduced into a cellular system at a predetermined location is a transgene or a cisgene or part of the transgene or cisgene of an organism of interest, wherein the transgene or the cisgene or part of the transgene or cisgene is selected from the group consisting of a gene encoding tolerance to abiotic stress, including drought stress, osmotic stress, heat stress, chilling stress, cold stress including frost, oxidative stress, heavy metal stress, nitrogen deficiency, phosphate deficiency, salt stress or waterlogging, herbicide resistance, including resistance to glyphosate, glufosinate/phosphinotricin, hygromycin (hyg), protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, bolting resistance, flowering time, shattering resistance, seed color, endosperm composition, or nutritional content.

In one embodiment according to the various aspects of the present invention, there is provided a method, wherein the at least one nucleic acid sequence of interest to be introduced into a cellular system at a predetermined location is at least part of a modified endogenous gene of an organism of interest, wherein the modified endogenous gene comprises at least one deletion, insertion and/or substitution of at least one nucleotide in comparison to the nucleic acid sequence of the unmodified (wildtype) endogenous gene.

In another embodiment according to the various aspects of the present invention, there is provided a method, wherein the at least one nucleic acid sequence of interest to be introduced into a cellular system at a predetermined location is at least part of a modified endogenous gene of an organism of interest, wherein the modified endogenous gene comprises at least one of a truncation, duplication, substitution and/or deletion of at least one nucleic acid position encoding a domain of the modified endogenous gene.

In yet another embodiment according to the various aspects of the present invention, there is provided a method, wherein the at least one nucleic acid sequence of interest to be introduced into a cellular system at a predetermined location is at least part of a regulatory sequence, wherein the regulatory sequence comprises at least one of a core promoter sequence, a proximal promoter sequence, a cis acting element, a trans acting element, a locus control sequences, an insulator sequence, a silencer sequence, an enhancer sequence, a terminator sequence, a conserved motif of a regulatory element like TATA box and/or any combination thereof.

In one embodiment according to the various aspects of the present invention, there is provided a method, wherein the at least one site-specific nuclease comprises a zinc-finger nuclease, a transcription activator-like effector nuclease, a CRISPR/Cas system, including a CRISPR/Cas9 system, a CRISPR/Cpf1 system, a CRISPR/CasX system, a CRISPR/CasY system, an engineered homing endonuclease, and a meganuclease, and/or any combination, variant, or catalytically active fragment thereof.

In a further embodiment according to the various aspects of the present invention, there is provided a method, wherein the one or more nucleic acid sequence(s) flanking the at least one nucleic acid sequence of interest at the predetermined location is/are at least 85%, 86%, 87%, 88%, or 89%, preferably at least 90%, 91%, 92%, 93%, 94% or 95%, more preferably at least 96%, 97%, 98%, 99%, 99.5% or 100% complementary to the one or more nucleic acid sequence(s) adjacent to the predetermined location, upstream and/or downstream from the predetermined location, over the entire length of the respective adjacent region(s).

In yet a further embodiment according to the various aspects of the present invention, there is provided a method, wherein the genetic material of the cellular system is selected from the group consisting of a protoplast, a viral genome transferred in a recombinant host cell, a eukaryotic or prokaryotic cell, tissue, or organ, and a eukaryotic or prokaryotic organism.

In one embodiment according to the various aspects of the present invention, there is provided a method, wherein the genetic material of the cellular system is selected from a eukaryotic cell, wherein the eukaryotic cell is a plant cell.

In a further embodiment according to the various aspects of the present invention, there is provided a method, wherein the eukaryotic organism is a plant, or a part of a plant.

In yet a further embodiment according to the various aspects of the present invention, there is provided a method, wherein the part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In one embodiment according to the various aspects of the present invention, there is provided a method, wherein the genetic material of the cellular system is, or originates from, a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

In a further aspect according to the present invention, there is provided a cellular system obtained by a method according to any one of the above aspects and embodiments.

In yet a further aspect according to the present invention, there is provided a cellular system comprising an inactivated or partially inactivated Polymerase theta (Pol theta) enzyme and one or more further inactivated or partially inactivated DNA repair enzyme(s) of a NHEJ pathway, wherein the modified cellular system is selected from the group consisting of one or more plant cell(s), a plant, and parts of a plant.

In one embodiment according to the various aspects disclosed herein, there is provided a cellular system, wherein the one or more part(s) of the plant is/are selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In another embodiment according to the various aspects disclosed herein, there is provided a cellular system, wherein the one or more plant cell(s), the plant(s) or the part(s) of a plant originate(s) from a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

Further aspects and embodiments of the present invention can be derived from the subsequent detailed description, the sequence listing as well as the attached set of claims.

### DRAWINGS

FIG. 1. Overview of PolQ, Ku70, Ku80 and LigIV gene expression in the mutants lines N698253 (teb-2), N667884 (teb-5), N656431 (ligIV), N656936 (ku70) and N677892 (ku80). Gene expression was determined by qRT-PCR using primers directed to a region not overlapping with the T-DNA insertion site. Col-0 wild type plants were used as reference. qRT-PCR data indicate that expression of PolQ, LigIV and Ku80 genes is significantly reduced in the respective mutant lines. Although Ku70 transcripts are detectable in N656936, the mutant line can be a null mutant.

FIG. 2. Depiction of the used gene targeting construct. LB/RB: Left border/right border; PcUbi4-2(P): Parsley ubiquitin promoter; Cas9: Cas9 nuclease; AtU6-26(P): U6 promter to express the guide RNA (sgRNA). The vertical lines indicate the recognition sites for the Cas9 nuclease, and mark the gene targeting cassette. The cassette is flanked by homologous sequences for the ADH1 gene target (674 bp upstream, 673 bp downstream) and a GFP coding sequence under control of the seed specific 2S promoter (A). Seed obtained after floral dip transformation of the targeting construct into Col-0 Arabidopsis plants. Right: bright field; Left: Green fluorescence. The white circles indicate fluorescent seeds (B).

FIG. 3. Bright field picture of transformed wildtype (Col-0) and mutant line teb-2. BASTA selection was done for aliquots of the transformed wildtype and mutant lines (shown is only the teb-2 mutant line. Results for the other mutant lines were similar). For none of the mutants BASTA resistant plants were identified, demonstrating that there is no random integration of the T-DNA into these mutants.

FIG. 4. Confirmation of gene targeting in fluorescent seeds by PCR. (A) #2: Fluorescent seed from transformed pol Q mutant line (putative gene targeting event); #3: Fluorescent seed from transformed Col-0 wild type plant (random integration). (B) PCR confirmation of gene targeting: #2, #3: DNA from plants grown from the respective fluorescent seeds. WT: DNA from untransformed Col-0 wildtype plant. P: Gene targeting vector (Plasmid DNA). PCR1: Wildtype adh1 locus. PCR2: Detection of the homologous recombination event using the primers HDRadh1-F (binding only in the adh1 genomic locus) and HDRadh1-R (binding in the 2S promoter of the gene targeting cassette). (C) Binding sites are indicated in the lower drawing, the product size is 945 bp. Formation of the product confirms a homologous recombination and is found only in fluorescent mutant seed (#2), while it is absent in the fluorescent wildtype seed. The Col-0 wildtype and the plasmid serve as controls. PCR3: Same as PCR2, except that primers HDRadh1-F2/R2 were used. These primers are binding a few bases upstream/downstream of the amplicon of PCR2, leading to a slight bigger product. PCR3 confirms the results of PCR2 with a second independent primer set.

### DEFINITIONS

The terms “associated with” or “in association with” according to the present disclosure are to be construed broadly and, therefore, according to the present invention imply that a molecule (DNA, RNA, amino acid, comprising naturally occurring and/or synthetic building blocks) is provided in physical association with another molecule, the association being either of covalent or non-covalent nature. For example, a repair template can be associated with a gRNA of a CRISPR nuclease, wherein the association can be of non covalent nature (complementary base pairing), or the molecules can be physically attached to each other by a covalent bond.

The term “catalytically active fragment” as used herein referring to amino acid sequences denotes the core sequence derived from a given template amino acid sequence, or a nucleic acid sequence encoding the same, comprising all or part of the active site of the template sequence with the proviso that the resulting catalytically active fragment still possesses the activity characterizing the template sequence, for which the active site of the native enzyme or a variant thereof is responsible. Said modifications are suitable to generate less bulky amino acid sequences still having the same activity as a template sequence making the catalytically active fragment a more versatile or more stable tool being sterically less demanding.

A “covalent attachment” or “covalent bond” is a chemical bond that involves the sharing of electron pairs between atoms of the molecules or sequences covalently attached to each other. A “non-covalent” interaction differs from a covalent bond in that it does not involve the sharing of electrons, but rather involves more dispersed variations of electromagnetic interactions between molecules/sequences or within a molecule/sequence. Non-covalent interactions or attachments thus comprise electrostatic interactions, van der Waals forces, π-effects and hydrophobic effects. Of special importance in the context of nucleic acid molecules are hydrogen bonds as electrostatic interaction. A hydrogen bond (H-bond) is a specific type of dipole-dipole interaction that involves the interaction between a partially positive hydrogen atom and a highly electronegative, partially negative oxygen, nitrogen, sulfur, or fluorine atom not covalently bound to said hydrogen atom. Any “association” or “physical association” as used herein thus implies a covalent or non-covalent interaction or attachment. In the case of molecular complexes, e.g. a complex formed by a CRISPR nuclease, a gRNA and a RT, more covalent and non-covalent interactions can be present for linking and thus associating the different components of a molecular complex of interest.

The terms “CRISPR polypeptide”, “CRISPR endonuclease”, “CRISPR nuclease”, “CRISPR protein”, “CRISPR effector” or “CRISPR enzyme” are used interchangeably herein and refer to any naturally occurring or artificial amino acid sequence, or the nucleic acid sequence encoding the same, acting as site-specific DNA nuclease or nickase, wherein the “CRISPR polypeptide” is derived from a CRISPR system of any organism, which can be cloned and used for targeted genome engineering. The terms “CRISPR nuclease” or “CRISPR polypeptide” also comprise mutants or catalytically active fragments or fusions of a naturally occurring CRISPR effector sequences, or the respective sequences encoding the same. A “CRISPR nuclease” or “CRISPR polypeptide” may thus, for example, also refer to a CRISPR nickase or even a nuclease-deficient variant of a CRISPR polypeptide having endonucleolytic function in its natural environment.

A “eukaryotic cell” as used herein refers to a cell having a true nucleus, a nuclear membrane and organelles belonging to any one of the kingdoms of Protista, Plantae, Fungi, or Animalia. Eukaryotic organisms can comprise monocellular and multicellular organisms. Preferred eukaryotic cells and organisms according to the present invention are plant cells (see below).

“Complementary” or “complementarity” as used herein describes the relationship between two (c)DNA, two RNA, or between an RNA and a (c)DNA nucleic acid region. Defined by the nucleobases of the DNA or RNA, two nucleic acid regions can hybridize to each other in accordance with the lock-and-key model. To this end the principles of Watson-Crick base pairing have the basis adenine and thymine/uracil as well as guanine and cytosine, respectively, as complementary bases apply. Furthermore, also non-Watson-Crick pairing, like reverse-Watson-Crick, Hoogsteen, reverse-Hoogsteen and Wobble pairing are comprised by the term “complementary” as used herein as long as the respective base pairs can build hydrogen bonding to each other, i.e. two different nucleic acid strands can hybridize to each other based on said complementarity.

The term “derivative” or “descendant” or “progeny” as used herein in the context of a prokaryotic or a eukaryotic cell, preferably an animal cell and more preferably a plant or plant cell or plant material according to the present disclosure relates to the descendants of such a cell or material which result from natural reproductive propagation including sexual and asexual propagation. It is well known to the person having skill in the art that said propagation can lead to the introduction of mutations into the genome of an organism resulting from natural phenomena which results in a descendant or progeny, which is genomically different to the parental organism or cell, however, still belongs to the same genus/species and possesses mostly the same characteristics as the parental recombinant host cell. Such derivatives or descendants or progeny resulting from natural phenomena during reproduction or regeneration are thus comprised by the term of the present disclosure and can be readily identified by the skilled person when comparing the “derivative” or “descendant” or “progeny” to the respective parent or ancestor. Furthermore, the term “derivative”, in the context of a substance or molecule and not referring to a replicating cell or organism, can imply a substance or molecule derived from the original substance or molecule by chemical and/or biotechnological means.

As used herein, “fusion” or “fused” can refer to a protein and/or nucleic acid comprising one or more non-native sequences (e.g., moieties). Any nucleic acid sequence or amino acid sequence according to the present invention can thus be provided in the form of a fusion molecule. A fusion can be at the N-terminal or C-terminal end of the modified protein, or both, or within the molecule as separate domain. For nucleic acid molecules, the fusion molecule can be attached at the 5′ or 3′ end, or at any suitable position in between. A fusion can be a transcriptional and/or translational fusion. A fusion can comprise one or more of the same non-native sequences. A fusion can comprise one 10 or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-specific effector or base editor (e.g., a nuclear localization signal (NLS) for targeting (e.g., a site-specific nuclease) to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as alexa fluor dyes, Cyanine3 dye, Cyanine5 dye. The fusion can provide for increased or decreased stability. In some embodiments, a fusion can comprise a detectable label, including a moiety that can provide a detectable signal. Suitable detectable labels and/or moieties that can provide a detectable signal can include, but are not limited to, an enzyme, a radioisotope, a member of a specific binding pair; a fluorophore; a fluorescent reporter or fluorescent protein; a quantum dot; and the like. A fusion can comprise a member of a FRET pair, or a fluorophore/quantum dot donor/acceptor pair. A fusion can comprise an enzyme. Suitable enzymes can include, but are not limited to, horse radish peroxidase, luciferase, beta-25 galactosidase, and the like. A fusion can comprise a fluorescent protein. Suitable fluorescent proteins can include, but are not limited to, a green fluorescent protein (GFP), (e.g., a GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum) any of a variety of fluorescent and colored proteins. A fusion can comprise a nanoparticle. Suitable nanoparticles can include fluorescent or luminescent nanoparticles, and magnetic nanoparticles, or nanodiamonds, optionally linked to a nanoparticle. Any optical or magnetic property or characteristic of the nanoparticle(s) can be detected. A fusion can comprise a helicase, a nuclease (e.g., FokI), an endonuclease, an exonuclease (e.g., a 5′ exonuclease and/or 3′ exonuclease), a ligase, a nickase, a nuclease-helicase (e.g., Cas3), a DNA methyltransferase (e.g., Dam), or DNA demethylase, a histone methyltransferase, a histone demethylase, an acetylase (including for example and not limitation, a histone acetylase), a deacetylase (including for example and not limitation, a histone deacetylase), a phosphatase, a kinase, a transcription (co-) activator, a transcription (co-) factor, an RNA polymerase subunit, a transcription repressor, a DNA binding protein, a DNA structuring protein, a long non-coding RNA, a DNA repair protein (e.g., a protein involved in repair of either single- and/or double-stranded breaks, e.g., proteins involved in base excision repair, nucleotide excision repair, mismatch repair, NHEJ, HR, microhomology-mediated end joining (MMEJ), and/or alternative non-homologous end-joining (ANHEJ), such as for example and not limitation, HR regulators and HR complex assembly signals), a marker protein, a reporter protein, a fluorescent protein, a ligand binding protein (e.g., mCherry or a heavy metal binding protein), a signal peptide (e.g., Tat-signal sequence), a targeting protein or peptide, a subcellular localization sequence (e.g., nuclear localization sequence, a chloroplast localization sequence), and/or an antibody epitope, or any combination thereof.

The terms “genetic construct” or “recombinant construct”, “vector”, or “plasmid (vector)” (e.g., in the context of at least one nucleic acid sequence to be introduced into a cellular system) are used herein to refer to a construct comprising, inter alia, plasmids or (plasmid) vectors, cosmids, artificial yeast- or bacterial artificial chromosomes (YACs and BACs), phagemides, bacterial phage based vectors, an expression cassette, isolated single-stranded or double-stranded nucleic acid sequences, comprising DNA and RNA sequences in linear or circular form, or amino acid sequences, viral vectors, including modified viruses, and a combination or a mixture thereof, for introduction or transformation, transfection or transduction into any prokaryotic or eukaryotic target cell, including a plant, plant cell, tissue, organ or material according to the present disclosure. “Recombinant” in the context of a biological material, e.g., a cell or vector, thus implies an artificially produced material. A recombinant construct according to the present disclosure can comprise an effector domain, either in the form of a nucleic acid or an amino acid sequence, wherein an effector domain represents a molecule, which can exert an effect in a target cell and includes a transgene, a cisgene, a single-stranded or double-stranded RNA molecule, including a guide RNA ((s)gRNA), a miRNA or an siRNA, or an amino acid sequences, including, inter alia, an enzyme or a catalytically active fragment thereof, a binding protein, an antibody, a transcription factor, a nuclease, preferably a site specific nuclease, and the like. Furthermore, the recombinant construct can comprise regulatory sequences and/or localization sequences. The recombinant construct can be integrated into a vector, including a plasmid vector, and/or it can be present isolated from a vector structure, for example, in the form of a polypeptide sequence or as a non-vector connected single-stranded or double-stranded nucleic acid. After its introduction, e.g. by transformation or transfection by biological or physical means, the genetic construct can either persist extrachromosomally, i.e. non integrated into the genome of the target cell, for example in the form of a double-stranded or single-stranded DNA, a double-stranded or single-stranded RNA or as an amino acid sequence. Alternatively, the genetic construct, or parts thereof, according to the present disclosure can be stably integrated into the genome of a target cell, including the nuclear genome or further genetic elements of a target cell, including the genome of plastids like mitochondria or chloroplasts. The term plasmid vector as used in this connection refers to a genetic construct originally obtained from a plasmid. A plasmid usually refers to a circular autonomously replicating extrachromosomal element in the form of a double-stranded nucleic acid sequence. In the field of genetic engineering these plasmids are routinely subjected to targeted modifications by inserting, for example, genes encoding a resistance against an antibiotic or an herbicide, a gene encoding a target nucleic acid sequence, a localization sequence, a regulatory sequence, a tag sequence, a marker gene, including an antibiotic marker or a fluorescent marker, a sequence, optionally encoding, a readily identifiable and the like. The structural components of the original plasmid, like the origin of replication, are maintained. According to certain embodiments of the present invention, the localization sequence can comprise a nuclear localization sequence (NLS), a plastid localization sequence, preferably a mitochondrion localization sequence or a chloroplast localization sequence. Said localization sequences are available to the skilled person in the field of plant biotechnology. A variety of plasmid vectors for use in different target cells of interest is commercially available and the modification thereof is known to the skilled person in the respective field.

A “genome” as used herein includes both the genes (the coding regions), the non-coding DNA and, if present, the genetic material of the mitochondria and/or chloroplasts, or the genomic material encoding a virus, or part of a virus. The “genome” or “genetic material” of an organism usually consists of DNA, wherein the genome of a virus may consist of RNA (single-stranded or double stranded).

The terms “genome editing”, “gene editing” and “genome engineering” are used interchangeably herein and refer to strategies and techniques for the targeted, specific modification of any genetic information or genome of a living organism at at least one position. As such, the terms comprise gene editing, but also the editing of regions other than gene encoding regions of a genome. It further comprises the editing or engineering of the nuclear (if present) as well as other genetic information of a cell. Furthermore, the terms “genome editing”, “gene editing” and “genome engineering” also comprise an epigenetic editing or engineering, i.e. the targeted modification of, e.g. methylation, histone modification or of non-coding RNAs possibly causing heritable changes in gene expression.

The terms “guide RNA”, “gRNA”, “single guide RNA”, or “sgRNA” are used interchangeably herein and either refer to a synthetic fusion of a CRISPR RNA (crRNA) and a trans-activating crRNA (tracrRNA), or the term refers to a single RNA molecule consisting only of a crRNA and/or a tracrRNA, or the term refers to a gRNA individually comprising a crRNA or a tracrRNA moiety. A tracr and a crRNA moiety, if present as required by the respective CRISPR polypeptide, thus do not necessarily have to be present on one covalently attached RNA molecule, yet they can also be comprised by two individual RNA molecules, which can associate or can be associated by non-covalent or covalent interaction to provide a gRNA according to the present disclosure. In the case of single RNA-guided endonucleases like Cpf1 (see Zetsche et al., 2015, supra), for example, a crRNA as single guide nucleic acid sequence might be sufficient for mediating DNA targeting.

The term “hybridization” as used herein refers to the pairing of complementary nucleic acids, i.e., DNA and/or RNA, using any process by which a strand of nucleic acid joins with a complementary strand through base pairing to form a hybridized complex. Hybridization and the strength of hybridization (i.e., the strength of the association between the nucleic acids) is impacted by such factors as the degree and length of complementarity between the nucleic acids, stringency of the conditions involved, the melting temperature (Tm) of the formed hybrid, and the G:C ratio within the nucleic acids. The term hybridized complex refers to a complex formed between two nucleic acid sequences by virtue of the formation of hydrogen bonds between complementary G and C bases and between complementary A and T/U bases. A hybridized complex or a corresponding hybrid construct can be formed between two DNA nucleic acid molecules, between two RNA nucleic acid molecules or between a DNA and an RNA nucleic acid molecule. For all constellations, the nucleic acid molecules can be naturally occurring nucleic acid molecules generated in vitro or in vivo and/or artificial or synthetic nucleic acid molecules. Hybridization as detailed above, e.g., Watson-Crick base pairs, which can form between DNA, RNA and DNA/RNA sequences, are dictated by a specific hydrogen bonding pattern, which thus represents a non-covalent attachment form according to the present invention. In the context of hybridization, the term “stringent hybridization conditions” should be understood to mean those conditions under which a hybridization takes place primarily only between homologous nucleic acid molecules. The term “hybridization conditions” in this respect refers not only to the actual conditions prevailing during actual agglomeration of the nucleic acids, but also to the conditions prevailing during the subsequent washing steps. Examples of stringent hybridization conditions are conditions under which primarily only those nucleic acid molecules that have at least 75%, preferably at least 80%, at least 85%, at least 90%, at least 91%, at least 92%, at least 93%, at least 94%, at least 95%, at least 96%, at least 97%, at least 98%, at least 99%, or at least 99.5% sequence identity undergo hybridization. Stringent hybridization conditions are, for example: 4×SSC at 65° C. and subsequent multiple washes in 0.1×SSC at 65° C. for approximately 1 hour. The term “stringent hybridization conditions” as used herein may also mean: hybridization at 68° C. in 0.25 M sodium phosphate, pH 7.2, 7% SDS, 1 mM EDTA and 1% BSA for 16 hours and subsequently washing twice with 2×SSC and 0.1% SDS at 68° C. Preferably, hybridization takes place under stringent conditions.

The terms “nucleotide” and “nucleic acid” with reference to a sequence or a molecule are used interchangeably herein and refer to a single- or double-stranded DNA or RNA of natural or synthetic origin. The term nucleotide sequence is thus used for any DNA or RNA sequence independent of its length, so that the term comprises any nucleotide sequence comprising at least one nucleotide, but also any kind of larger oligonucleotide or polynucleotide. The term(s) thus refer to natural and/or synthetic deoxyribonucleic acids (DNA) and/or ribonucleic acid (RNA) sequences, which can optionally comprise synthetic nucleic acid analoga. A nucleic acid according to the present disclosure can optionally be codon optimized. Codon optimization implies that the codon usage of a DNA or RNA is adapted to that of a cell or organism of interest to improve the transcription rate of said recombinant nucleic acid in the cell or organism of interest. The skilled person is well aware of the fact that a target nucleic acid can be modified at one position due to the codon degeneracy, whereas this modification will still lead to the same amino acid sequence at that position after translation, which is achieved by codon optimization to take into consideration the species-specific codon usage of a target cell or organism. Nucleic acid sequences according to the present application can carry specific codon optimization for the following non limiting list of organisms: Hordeum vulgare, Sorghum bicolor, Secale cereale, Triticale, Saccharum officinarium, Zea mays, Setaria italic, Oryza sativa, Oryza minuta, Oryza australiensis, Oryza alta, Triticum aestivum, Triticum durum, Hordeum bulbosum, Brachypodium distachyon, Hordeum marinum, Aegilops tauschii, Malus domestica, Beta vulgaris, Helianthus annuus, Daucus glochidiatus, Daucus pusillus, Daucus muricatus, Daucus carota, Eucalyptus grandis, Erythranthe guttata, Genlisea aurea, Nicotiana sylvestris, Nicotiana tabacum, Nicotiana tomentosiformis, Nicotiana benthamiana, Solanum lycopersicum, Solanum tuberosum, Coffea canephora, Vitis vinifera, Cucumis sativus, Morus notabilis, Arabidopsis thaliana, Arabidopsis lyrata, Arabidopsis arenosa, Crucihimalaya himalaica, Crucihimalaya wallichfi, Cardamine flexuosa, Lepidium virginicum, Capsella bursa-pastoris, Olmarabidopsis pumila, Arabis hirsuta, Brassica napus, Brassica oleracea, Brassica rapa, Brassica juncacea, Brassica nigra, Raphanus sativus, Eruca vesicaria sativa, Citrus sinensis, Jatropha curcas, Glycine max, Gossypium ssp., Populus trichocarpa, Mus musculus, Rattus norvegicus or Homo sapiens.

The term “particle bombardment” as used herein, also named “biolistic transfection” or “biolistic bombardment” or “microparticle-mediated gene transfer”, refers to a physical delivery method for transferring a coated microparticle or nanoparticle comprising a nucleic acid or a genetic construct of interest into a target cell or tissue. The micro- or nanoparticle functions as projectile and is fired on the target structure of interest under high pressure using a suitable device, often called “gene-gun”. The transformation via particle bombardment uses a microprojectile of metal covered with the gene of interest, which is then shot onto the target cells using an equipment known as “gene-gun” (Sandford et al. 1987) at high velocity fast enough to penetrate the cell wall of a target tissue, but not harsh enough to cause cell death. For protoplasts, which have their cell wall entirely removed, the conditions are different logically. The precipitated nucleic acid or the genetic construct on the at least one microprojectile is released into the cell after bombardment, and integrated into the genome or expressed transiently according to the definition given above. The acceleration of microprojectiles is accomplished by a high voltage electrical discharge or compressed gas (helium). Concerning the metal particles used it is mandatory that they are non-toxic, non-reactive, and that they have a smaller diameter than the target cell. The most commonly used are gold or tungsten. There is plenty of information publicly available from the manufacturers and providers of gene-guns and associated system concerning their general use.

The terms “plant” or “plant cell” as used herein refer to a plant organism, a plant organ, differentiated and undifferentiated plant tissues, plant cells, seeds, and derivatives and progeny thereof. Plant cells include without limitation, for example, cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae. The different eukaryotic cells, for example, animal cells, fungal cells or plant cells, can have any degree of ploidity, i.e. they may either be haploid, diploid, tetraploid, hexaploid or polyploid.

The term “regulatory sequence” or “regulatory element” as used herein refers to a nucleic acid or an amino acid sequence, which can direct the transcription and/or translation and/or modification of a nucleic acid sequence of interest in a genome or genetic material of interest, either in cis or in trans. Such elements may include promoters, including core promoter elements or core promoter motifs, leader sequences, enhancers, silencer elements, introns, transcription termination regions (terminators), and untranslated regions upstream and downstream of a coding sequence. A “regulatory sequence” as understood according to the present disclosure may thus also comprise a part of a regulatory sequence or a regulatory element, which can influence, i.e., up- or down-regulate or shut-off, the activity of a native regulatory sequence or element, when introduced into a given regulatory sequence or element.

The terms “RNA interference” or “RNAi” as used herein interchangeably refer to a gene down-regulation mechanism meanwhile demonstrated to exist in all eukaryotes. The mechanism was first recognized in plants where it was called “post-transcriptional gene silencing” or “PTGS”. In RNAi, small RNAs (of about 21-24 nucleotides) function to guide specific effector proteins (e.g., members of the Argonaute protein family) to a target nucleotide sequence by complementary base pairing. The effector protein complex then down-regulates the expression of the targeted RNA or DNA. Small RNA-directed gene regulation systems were independently discovered (and named) in plants, fungi, worms, flies, and mammalian cells. Collectively, PTGS, RNA silencing, and co-suppression (in plants); quelling (in fungi and algae); and RNAi (in Caenorhabditis elegans, Drosophila, and mammalian cells) are all examples of small RNA-based gene regulation systems.

A “site-specific nuclease” or “SSN” as used herein refers to at least one usually genetically engineered nuclease or a catalytically active fragment thereof, or the corresponding sequence encoding the same, which acts as an enzyme catalyzing a site-specific and not random double stand break (DSB) or a single strand nick at a desired location of a genome or genomic sequence of interest in a precise way. DNA binding, recognition and cleavage capabilities of the SSNs according to the present disclosure may vary depending on the functional class of a SSN of interest.

A “transgene” or “transgenic sequence” as used herein refers to a gene, or part of a gene including the regulatory sequences thereof and introns, which has been artificially transferred from a donor genome to an acceptor genome or system. A “transgenic sequence” may thus be understood as a sequence foreign to the species the acceptor cell or genome belongs to.

A “cisgene” or “cisgenic sequence” as used herein refers to a gene, or part of a gene including the regulatory sequences thereof and introns, which has been artificially transferred from a donor genome to an acceptor genome or system. A “cisgenic sequence” may thus be understood as a sequence from the same species being transferred to another individual of the same species or to another cell of the same species.

The terms “transient” or “transient introduction” as used herein refer to the transient introduction of at least one nucleic acid and/or amino acid sequence according to the present disclosure, preferably incorporated into a delivery vector and/or into a recombinant construct, with or without the help of a delivery vector, into a target structure, for example, a plant cell, wherein the at least one nucleic acid sequence is introduced under suitable reaction conditions so that no integration of the at least one nucleic acid sequence into the endogenous nucleic acid material of a target structure, the genome as a whole, occurs, so that the at least one nucleic acid sequence will not be integrated into the endogenous DNA of the target cell. As a consequence, in the case of transient introduction, the introduced genetic construct will not be inherited to a progeny of the target structure, for example a prokaryotic, an animal or a plant cell. The at least one nucleic acid and/or amino acid sequence or the products resulting from transcription, translation, processing, post-translational modifications or complex building thereof are only present temporarily, i.e., in a transient way, in constitutive or inducible form, and thus can only be active in the target cell for exerting their effect for a limited time. Therefore, the at least one sequence introduced via transient introduction will not be heritable to the progeny of a cell. The effect mediated by at least one sequence or effector introduced in a transient way can, however, potentially be inherited to the progeny of the target cell.

A “variant” of any site-specific nuclease disclosed herein represents a molecule comprising at least one mutation, deletion or insertion in comparison to the wild-type site-specific nuclease to alter the activity of the wild-type nuclease as naturally occurring. A “variant” can, as non-limiting example, be a catalytically inactive Cas9 (dCas9), or a site-specific nuclease, which has been modified to function as nickase.

Whenever the present disclosure relates to the percentage of identity of nucleic acid or amino acid sequences to each other these values define those values as obtained by using the EMBOSS Water Pairwise Sequence Alignments (nucleotide) programme (www.ebi.ac.uk/Tools/psa/emboss_water/nucleotide.html) nucleic acids or the EMBOSS Water Pairwise Sequence Alignments (protein) programme (www.ebi.ac.uk/Tools/psa/emboss_water/) for amino acid sequences. Alignments or sequence comparisons as used herein refer to an alignment over the whole length of two sequences compared to each other. Those tools provided by the European Molecular Biology Laboratory (EMBL) European Bioinformatics Institute (EBI) for local sequence alignments use a modified Smith-Waterman algorithm (see www.ebi.ac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

### DETAILED DESCRIPTION

The multi-step NHEJ pathway is mediated by a number of highly conserved enzymes required for completion of double-strand break (DSB) repair by this mechanism. Knock-outs or knock-downs of any of these essential enzymes impair the ability of cells to use the NHEJ pathway. Impaired function of NHEJ tends to favor HDR as a partially compensatory mechanism to preserve a cell's aim to achieve chromosomal integrity in the presence of DSBs.

The present invention is thus in part based on the discovery that cells or cellular systems showing inhibited expression of POLQ and one of several enzymes essential for NHEJ repair (e.g., LigIV, Ku70, Ku80 and further enzymes disclosed herein) just simultaneously when performing targeted genome editing (GE) in exactly this cell or cellular system exhibit dominance of HR-mediated DSB repair with no random integration of supplied repair template(s) (RT). The findings on relevant NHEJ/H(D)R players and their inhibition were combined with and exploited for highly efficient gene targeting, as the absence of random RT integration and of NHEJ-mediated DSB repair guarantees a significantly improved precision and predictability of any GE experiment, in particular in eukaryotic cells and systems. The present invention thus provides methods to perform a targeted NHEJ pathway knock-out or knock-down simultaneous with performing GE so that it can be assured that NHEJ enzymes responsible for imprecise DSB repair after a DSB break will not be active in one cell or cellular system of interest, exactly at the time point a GE event including DSB and repair is to be effected in said one cell or cellular system.

The present invention discloses methods for efficient gene targeting in cells, preferably eukaryotic cells, and more preferably plant cells. Fundamentally, the methods rely on the provision of a reduced or abolished expression of Pol theta and at least one further enzyme essential for NHEJ repair which allows to perform gene targeting in a highly precise manner in one and the same cell. In a cell or a cellular system in which the enzyme Pol theta and at least one further NHEJ enzyme are (partially) inactivated, genomic double-strand breaks are predominantly repaired by HR. Such a cell or cellular system will thus allow for highly predictable Gene Editing when transformed with an RT.

In a first aspect, there is thus provided a method for modifying the genetic material of a cellular system at a predetermined location with at least one nucleic acid sequence of interest, wherein the method comprises the following steps: (a) providing a cellular system comprising a Polymerase theta enzyme, or a sequence encoding the same, and one or more further enzyme(s) of a NHEJ pathway, or the sequence(s) encoding the same; (b) inactivating or partially inactivating the Polymerase theta enzyme, or the sequence encoding the same, and inactivating or partially inactivating the one or more further DNA repair enzyme(s) of a NHEJ pathway, or the sequence(s) encoding the same; (c) introducing into the cellular system or a progeny system thereof (i) the at least one nucleic acid sequence of interest, optionally flanked by one or more homology sequence(s) complementary to one or more nucleic acid sequence(s) adjacent to the predetermined location, and (ii) at least one site-specific nuclease, or a sequence encoding the same, the site-specific nuclease inducing a double-strand break at the predetermined location; and (d) optionally: determining the presence of the modification at the predetermined location in the genetic material of the cellular system; (e) obtaining a cellular system comprising a modification at the predetermined location of the genetic material of the cellular system or selecting a cellular system comprising a modification at the predetermined location of the genetic material of the cellular system based on the determination of (d).

Notably, in one embodiment, steps (b) and (c) may be performed simultaneous. Depending on the mode of inactivation or partial inactivation as disclosed in step (b) of the above aspect, step (b) may be performed before step (c). Vice versa step (c) can also be performed before step (b). In one embodiment, the introduction of at least one nucleic acid sequence of interest and the introduction of at least one site-specific nuclease, or a sequence encoding the same may be performed simultaneously or in any sequential order in relation to each other and further in relation to the step of inactivation or partial inactivation of Polymerase theta enzyme, or a sequence encoding the same, and/or one or more further enzyme(s) of a NHEJ pathway, or the sequence(s) encoding the same. The sequential and temporal order of method steps will depend on the nature of the material to be introduced and the mode of inactivation, respectively. For example, when performing a knock-out or inactivation of the Polymerase theta enzyme, and/or the one or more further enzyme(s) of a NHEJ pathway this step will likely precede the subsequent method steps. In other embodiments, a transient (partial) inactivation may be more suitable. In this embodiment, step (b) can be conducted simultaneously with, or temporally even after any one of steps (c)(i) or (c)(ii) is performed.

For all aspects and embodiments according to the present invention it is of importance that the (partial) inactivation as detailed in step (b) of the first aspect of the present invention and the introduction of at least one site-specific nuclease, or a sequence encoding the same, is planned in a manner so that it can be guaranteed that one and the same cell, or one and the same cellular system comprising the genetic material to be modified will simultaneously comprise both, A) the (partially) inactivated Pol theta and the at least one further (partially) inactivated NHEJ enzyme as well as B) the (active) at least one site-specific nuclease and the at least one nucleic acid sequence of interest in one and the same cell or cellular system to achieve a significantly improved and more precise GE, as the imprecise NHEJ pathway will be (partially) inactivated in a spatio-temporal manner so that GE can be performed without inserting unwanted nucleotides at the site of a DSB induced in a targeted way.

The main contribution of the present invention is thus the provision of methods and the material as obtained by said methods, wherein NHEJ pathways significantly hampering a targeted GE event mediated by HDR are (partially) inactivated exactly at the time point and in the same cellular system and compartment thereof needed, when inducing GE to obtain optimum GE results without an undesired outcome.

A “modification” or “modifying” a genetic material according to the present disclosure implies any kind of insertion, deletion, and/or replacement of at least one nucleic acid sequence of interest effected at a predetermined location in a genome or a genetic material of interest.

A “cellular system” as used herein refers to at least one element comprising all or part of the genome of a cell of interest to be modified. The cellular system may thus be any in vivo or in vitro system, including also a cell-free system. The cellular system thus comprises and provides the target genome or genomic sequence to be modified in a suitable way, i.e., in a form accessible to a genetic modification or manipulation. The cellular system may thus be selected from, for example, a prokaryotic or eukaryotic cell, including an animal or a plant cell, a prokaryotic or eukaryotic organism, including an animal or plant, or the cellular system may comprise a genetic construct as defined above comprising all or parts of the genome of a prokaryotic or eukaryotic cell to be modified in a highly targeted way. The cellular system may be provided as isolated cell or vector, or the cellular system may be comprised by a network of cells in a tissue, organ, material or whole organism, either in vivo or as isolated system in vitro. In this context, the “genetic material” of a cellular system can thus be understood as all, or part of the genome of an organism the genetic material of which organism as a whole or in part is present in the cellular system to be modified.

In one aspect, the present invention provides a cellular system which may be obtained by a method according to any one of the above aspects and embodiments.

In one embodiment, the cellular system may comprise an inactivated or partially inactivated Polymerase theta (Pol theta) enzyme and one or more further inactivated or partially inactivated DNA repair enzyme(s) of a NHEJ pathway, wherein the modified cellular system may be selected from the group consisting of one or more plant cell(s), a plant, and parts of a plant.

A “partial” inactivation in this context implies a reduced activity of the Pol theta and/or of the further DNA repair enzyme(s) of a NHEJ pathway in comparison to the enzymatic activity of the respective wild-type enzyme not partially inactivated measured under the same conditions in vivo or in vitro. An “inactivation” thus implies a complete, or almost complete, loss of enzymatic activity. Partial and full inactivation may be temporally limited. According to the present invention, the relevant time point for providing a state of a (partial) inactivation is the time point when GE including DSB induction and targeted repair is performed.

In one embodiment according to the various aspects disclosed herein for providing a cellular system comprising a modified genetic material, the one or more part(s) of the plant may be selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In another embodiment according to the various aspects disclosed herein, there is provided a cellular system, wherein the one or more plant cell(s), the plant(s) or the part(s) of a plant may originate from a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum.

A “homology sequence”, if present, may be part of the at least one nucleic acid sequence of interest according to the various embodiments of the present invention, to be introduced to modify the genetic material of a cellular system according to the present disclosure. Therefore, the at least one homology sequence is physically associated with the at least one nucleic acid sequence of interest within one molecule. As such, the homology sequence may be part of the at least one nucleic acid sequence of interest to be introduced and it may be positioned within the 5′ and/or 3′ position of the at least one nucleic acid sequence of interest, optionally including at least one spacer nucleotide, or within the at least one nucleic acid sequence of interest to be introduced. As such, the homology sequence(s) serve as templates to mediate homology-directed repair by having complementarity to at least one region, the upstream and/or the downstream region, adjacent to the predetermined location within the genetic material of the cellular system to be modified. The at least one nucleic acid sequence of interest and the flanking one or more homology region(s) thus can have the function of a repair template (RT) nucleic acid sequence. In certain embodiments, the RT may be further associated with another DNA and/or RNA sequence as mediated by complementary base pairing. In an alternative embodiment the RT may be associated with other sequence, for example, sequences of a vector, e.g., a plasmid vector, which vector can be used to amplify the RT prior to transformation. Furthermore, the RT may also be physically associated with at least part of an amino acid component, preferably a site-specific nuclease. This configuration and association allows the availability of the RT in close physical proximity to the site of a DSB, i.e., exactly at the position a targeted GE event is to be effected to allow even higher efficiency rates. For example, the at least one RT may also be associated with at least one gRNA interacting with the at least one RT and further interacting with at least one portion of a CRISPR nuclease as site-specific nuclease.

The one or more homology region(s) will each have a certain degree of complementarity to the respective region flanking the at least one predetermined location upstream and/or downstream of the double-strand break induced by the at least one site-specific nuclease, i.e., the upstream and downstream adjacent region, respectively. Preferably, the one or more homology region(s) will hybridize to the upstream and/or downstream adjacent region under conditions of high stringency. The longer the at least one homology region, the lower the degree of complementarity may be. The complementarity is usually calculated over the whole length of the respective region of homology. In case only one homology region is present, this single homology region will usually have a higher degree of complementarity to allow hybridization. Complementarity under stringent hybridization conditions will be at least 85%, at least 90%, at least 91%, at least 92%, at least 93%, at least 94%, at least 95%, at least 96%, and preferably at least 97%, at least 98%, at least 99%, at least 99.5% or even 100%. At least in the region directly flanking a DSB induced (about 5 to 10 bp upstream and downstream of a DSB), complementarities of at least 98%, at least 99%, at least 99.5% and preferably 100% should be present. Notably, as further disclosed herein below, the degree of complementarity can also be lower than 85%. This will largely depend on the target genetic material and the complexity of the genome it is derived from, the length of the nucleic acid sequence of interest to be introduced, the length and nature of the further homology arm or flanking region, the relative position and orientation of the flanking region in relation to the site at least one DSB is induced, and the like.

The term “adjacent” or “adjacent to” as used herein in the context of the predetermined location and the one or more homology region(s) may comprise an upstream and a downstream adjacent region, or both. Therefore, the adjacent region is determined based on the genetic material of a cellular system to be modified, said material comprising the predetermined location.

There may be an upstream and/or downstream adjacent region near the predetermined location. For site-specific nucleases (SSNs) inducing blunt double-strand breaks (DSBs), the “predetermined location” will represent the site the DSB is induced within the genetic material in a cellular system of interest. For SSNs leaving overhangs after DSB induction, the predetermined location means the region between the cut in the 5′ end on one strand and the 3′ end on the other strand. The adjacent regions in the case of sticky end SSNs thus may be calculated using the two different DNA strands as reference. The term “adjacent to a predetermined location” thus may imply the upstream and/or downstream nucleotide positions in a genetic material to be modified, wherein the adjacent region is defined based on the genetic material of a cellular system before inducing a DSB or modification. Based on the different mechanisms of SSNs inducing DSBs, the “predetermined location” meaning the location a modification is made in a genetic material of interest may thus imply one specific position on the same strand for blunt DSBs, or the region on different strands between two cut sites for sticky cutting DSBs, or for nickases used as SSNs between the cut at the 5′ position in one strand and at the 3′ position in the other strand.

If present, the upstream adjacent region defines the region directly upstream of the 5′ end of the cutting site of a site-specific nuclease of interest with reference to a predetermined location before initiating a double-strand break, e.g., during targeted genome engineering. Correspondingly, a downstream adjacent region defines the region directly downstream of the 3′ end of the cutting site of a SSN of interest with reference to a predetermined location before initiating a double-strand break, e.g., during targeted genome engineering. The 5′ end and the 3′ end can be the same, depending on the site-specific nuclease of interest.

In certain embodiments, it may also be favorable to design at least one homology region in a distance away from the DSB to be induced, i.e., not directly flanking the predetermined location/the DSB site. In this scenario, the genomic sequence between the predetermined location and the homology sequence (the homology arm) would be “deleted” after homologous recombination had occurred, which may be preferred for certain strategies as this allows the targeted deletion of sequences near the DSB. Different kinds of RT configuration and design are thus contemplated according to the present invention for those embodiments relying on a RT. RTs may be used to introduce site-specific mutations, or RTs may be used for the site-specific integration of nucleic acid sequences of interest, or RTs may be used to assist a targeted deletion.

A “homology sequence(s)” introduced and the corresponding “adjacent region(s)” can each have varying and different length from about 15 bp to about 15.000 bp, i.e., an upstream homology region can have a different length in comparison to a downstream homology region. Only one homology region may be present. There is no real upper limit for the length of the homology region(s), which length is rather dictated by practical and technical issues. According to certain embodiments, depending on the nature of the RT and the targeted modification to be introduced, asymmetric homology regions may be preferred, i.e., homology regions, wherein the upstream and downstream flanking regions have varying length. In certain embodiments, only one upstream and downstream flanking region may be present.

Based on the above, a “predetermined location” according to the present invention means the location or site in a genetic material in a cellular system, or within a genome of a cell of interest to be modified, where a targeted edit or modification is to be introduced. In certain embodiments, the predetermined location may thus coincide with the DSB induced by the at least one site-specific nuclease, wherein in other embodiments, the predetermined location may comprise the site of the DSB induced without directly aligning with the cut sites of the at least one site-specific nuclease. In yet a further embodiment, the predetermined location may be away from, i.e., at a certain distance to the DSB site. Depending on the nature of the modification to be introduced this may be the case for embodiments, wherein a RT is used comprising at least one homology region aligning at a certain distance from the site of a DSB induced, or spanning the DSB site, and not directly aligning with the upstream and the downstream region of an induced DSB.

In one embodiment according to the various aspects of the present invention, the method may comprise an additional step of: (f) restoring the activity of the inactivated or partially inactivated Polymerase theta enzyme and/or restoring the activity of the one or more further inactivated or partially inactivated DNA repair enzyme(s) of a NHEJ pathway in the cellular system comprising a modification at the predetermined location, or in a progeny system thereof.

Restoration of the at least one NHEJ enzyme (partially) inactivated may be advantageous to provide a cellular system, a cell, a tissue, an organ, or a whole organism, preferably a plant or an animal, wherein the natural NHEJ pathways are fully active to fulfill their inherent functions in naturally occurring DNA damage to preserve genome integrity. It has to be emphasized that in certain embodiments according to the present invention, the cellular systems or the cell to be modified, i.e. the cell, where at least one NHEJ pathway is (partially) inactivated exactly when a GE event is introduced, will have the capacity to be cultivated, or to develop into an organism. In particular for embodiments, wherein the cellular system is, or is derived from a plant cell, including cells from seeds, from mature and immature embryos, meristematic tissues, seedlings, callus tissues in different differentiation states, leaves, flowers, roots, shoots, male or female gametophytes, sporophytes, pollen, pollen tubes and microspores, protoplasts, macroalgae and microalgae, wherein the different plant cells can have any degree of ploidity, i.e. they may either be haploid, diploid, tetraploid, hexaploid or polyploidy, the cellular system modified according to the present invention will be used to develop a whole plant organism. Using techniques known to the skilled person, a plant can be crossed with other plants to possibly restore the activity of at least one Pol theta enzyme and/or the activity of at least one further NHEJ pathway enzyme using suitable breeding strategies.

In one embodiment according to the various aspects of the present invention, the Polymerase theta to be inactivated or partially inactivated may comprise an amino acid sequence according to SEQ ID NO: 2, 7, 8, 9 or 10, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 2, 7, 8, 9 or 10, respectively, preferably over the entire length of the sequence; or it may be encoded by the nucleic acid sequence according to SEQ ID NO: 1, 3, 4, 5 or 6, or a nucleic acid having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID No: 1, 3, 4, 5 or 6, respectively, preferably over the entire length of the sequence; or it may be encoded by a nucleic acid sequence hybrizing to a nucleic acid sequence complementary to the nucleic acid sequence according to SEQ ID NO: 1, 3, 4, 5 or 6 under stringent conditions.

In yet a further embodiment according to the various aspects of the present invention, the one or more further DNA repair enzyme(s) of a NHEJ pathway to be inactivated or partially inactivated may be independently selected from the group consisting of Ku70, Ku80, DNA-dependent protein kinase, Ataxia telangiectasia mutated (ATM), ATM—and Rad3—related (ATR), Artemis, XRCC4, DNA ligase IV (LigIV) and XLF, or any combination thereof.

In one embodiment according to the various aspects of the present invention, at least one, at least two, at least three, or at least four further DNA repair enzymes of a NHEJ pathway may be inactivated or partially inactivated, preferably wherein at least Ku70 and DNA ligase IV, or wherein at least Ku80 and DNA ligase IV may be inactivated or partially inactivated.

In another embodiment according to the various aspects of the present invention, one, two, three, or four, preferably solely one, solely two, solely three or solely four, further DNA repair enzymes of a NHEJ pathway may be inactivated or partially inactivated, preferably wherein the Ku70 and DNA ligase IV, or wherein the Ku80 and DNA ligase IV may be inactivated or partially inactivated.

In one embodiment according to the various aspects of the present invention, the one or more further DNA repair enzyme(s) of a NHEJ pathway to be inactivated or partially inactivated may be Ku70, or a nucleic acid sequence encoding the same, wherein the Ku70 may comprise an amino acid sequence according to SEQ ID NO: 12, 18, 19 or 20, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 12, 18, 19 or 20, respectively, preferably over the entire length of the sequence, or the nucleic acid sequence encoding the same may comprise a nucleic acid sequence according to SEQ ID NO: 11, 13, 14, 15, 16 or 17, or may comprise a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 11, 13, 14, 15, 16 or 17, respectively, preferably over the entire length of the sequence, or may comprise a nucleic acid sequence hybridizing to a nucleic acid sequence complementary to the nucleic acid sequence according to SEQ ID NO: 11, 13, 14, 15, 16 or 17.

In a further embodiment, wherein the one or more further DNA repair enzyme(s) of a NHEJ pathway to be inactivated or partially inactivated may be Ku80, or a nucleic acid sequence encoding the same, wherein the Ku80 may comprise an amino acid sequence according to SEQ ID NO: 22, 23, 24 or 29, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 22, 23, 24 or 29, respectively, preferably over the entire length of the sequence, or the nucleic acid sequence encoding the same may comprise a sequence according to SEQ ID NO: 21, 25, 26, 27 or 28, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 21, 25, 26, 27 or 28, respectively, preferably over the entire length of the sequence, or may comprise a nucleic acid sequence hybridizing to a nucleic acid sequence complementary to the nucleic acid sequence according to SEQ ID NO: 21, 25, 26, 27 or 28.

In a further embodiment, wherein the one or more further DNA repair enzyme(s) of a NHEJ pathway to be inactivated or partially inactivated may be DNA-dependent protein kinase, or a nucleic acid sequence encoding the same, the DNA-dependent protein kinase may comprise an amino acid sequence according to SEQ ID NO: 32, 33 or 35, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 32, 33 or 35, respectively, preferably over the entire length of the sequence, or the nucleic acid sequence encoding the same may comprise a sequence according to SEQ ID NO: 30, 31 or 34, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 30, 31 or 34, respectively, preferably over the entire length of the sequence, or may comprise a nucleic acid sequence hybridizing to a nucleic acid sequence complementary to the nucleic acid sequence according to SEQ ID NO: 30, 31 or 34.

In yet a further embodiment, wherein the one or more further DNA repair enzyme(s) of a NHEJ pathway to be inactivated or partially inactivated may be ATM, or a nucleic acid sequence encoding the same, the ATM may comprise an amino acid sequence according to SEQ ID NO: 37, 38, 39, 41, 42, 43, 44, 45, 46, 47 or 48, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 37, 38, 39, 41, 42, 43, 44, 45, 46, 47 or 48, respectively, preferably over the entire length of the sequence, or the nucleic acid sequence encoding the same may comprise a sequence according to SEQ ID NO: 36 or 40, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 36 or 40, respectively, preferably over the entire length of the sequence, or may comprise a nucleic acid sequence hybridizing to a nucleic acid sequence complementary to the nucleic acid sequence according to SEQ ID NO: 36 or 40.

In still a further embodiment, wherein the one or more further DNA repair enzyme(s) of a NHEJ pathway to be inactivated or partially inactivated may be ATM—and Rad3—related (ATR), or a nucleic acid sequence encoding the same, the ATR may comprise an amino acid sequence according to SEQ ID NO: 50, 51, 52, 53, 55 or 56, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 50, 51, 52, 53, 55 or 56, respectively, preferably over the entire length of the sequence, or the nucleic acid sequence encoding the same may comprise a sequence according to SEQ ID NO: 49 or 54, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 49 or 54, respectively, preferably over the entire length of the sequence, or may comprise a nucleic acid sequence hybridizing to a nucleic acid sequence complementary to the nucleic acid sequence according to SEQ ID NO: 49 or 54.

In a further embodiment, wherein the one or more further DNA repair enzyme(s) of a NHEJ pathway to be inactivated or partially inactivated may be Artemis, or a nucleic acid sequence encoding the same, the Artemis may comprise an amino acid sequence according to SEQ ID NO: 60, 61, 62 or 64, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 60, 61, 62 or 64, respectively, preferably over the entire length of the sequence, or the nucleic acid sequence encoding the same may comprise a sequence according to SEQ ID NO: 57, 58, 59 or 63, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 57, 58, 59 or 63, respectively, preferably over the entire length of the sequence, or may comprise a nucleic acid sequence hybridizing to a nucleic acid sequence complementary to the nucleic acid sequence according to SEQ ID NO: 57, 58, 59 or 63.

In another embodiment, wherein the one or more further DNA repair enzyme(s) of a NHEJ pathway to be inactivated or partially inactivated may be XRCC4, or a nucleic acid sequence encoding the same, the XRCC4 may comprise an amino acid sequence according to SEQ ID NO: 66, 67, or 69, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 66, 67 or 69, respectively, preferably over the entire length of the sequence, or the nucleic acid sequence encoding the same may comprise a sequence according to SEQ ID NO: 65 or 68, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 65 or 68, respectively, preferably over the entire length of the sequence, or may comprise a nucleic acid sequence hybridizing to a nucleic acid sequence complementary to the nucleic acid sequence according to SEQ ID NO: 65 or 68.

In a further embodiment, wherein the one or more further DNA repair enzyme(s) of a NHEJ pathway to be inactivated or partially inactivated may be DNA ligase IV, or a nucleic acid sequence encoding the same, the DNA ligase IV may comprise an amino acid sequence according to SEQ ID NO: 71, 72, 76 or 77, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 71, 72, 76 or 77, respectively, preferably over the entire length of the sequence, or the nucleic acid sequence encoding the same may comprise a sequence according to SEQ ID NO: 70, 73, 74 or 75, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 70, 73, 74 or 75, respectively, preferably over the entire length of the sequence, or may comprise a nucleic acid sequence hybridizing to a nucleic acid sequence complementary to the nucleic acid sequence according to SEQ ID NO: 70, 73, 74 or 75.

In still another embodiment, the one or more further DNA repair enzyme(s) of a NHEJ pathway to be inactivated or partially inactivated may be XLF, or a nucleic acid sequence encoding the same.

In certain embodiments, a transient knock-down of at least one Pol theta and the at least one further enzyme of a NHEJ pathway may be preferable, for example, for certain NHEJ enzymes being deleterious to a cell in the homozygous knocked-out stage, so that a transient down-regulation to effect a targeted GE followed by a restoration of the activity of the at least one NHEJ enzyme and/or the Pol theta functionality may be desirable.

In one embodiment according to the various aspects of the present invention, the at least one nucleic acid sequence of interest may be provided as part of at least one vector, or as at least one linear molecule. In another aspect, the at least one nucleic acid sequence of interest may be provided as a complex, preferably a complex physically associating with the at least one nucleic acid sequence and another RT, and/or with a gRNA, and/or with a site-specific nuclease. The at least one nucleic acid sequence of interest may further comprise a sequence allowing the rapid traceability, including the visual traceability, of the sequence of interest, e.g., a tag, including a fluorescent tag. The at least one nucleic acid sequence of interest may be double-stranded, single-stranded, or a mixture thereof. Furthermore, the at least one nucleic acid sequence of interest may comprise a mixture of DNA and RNA nucleotide, including also synthetic, i.e., non-naturally occurring nucleotides.

In another embodiment according to the various aspects of the present invention, the at least one vector used according to the various methods disclosed herein may be introduced into the cellular system by biological or physical means, including transfection, transformation, including transformation by Agrobacterium spp., preferably by Agrobacterium tumefaciens, a viral vector, biolistic bombardment, transfection using chemical agents, including polyethylene glycol transfection, or any combination thereof.

Further provided is an embodiment of the methods according to the various aspects disclosed herein, wherein the at least one site-specific nuclease or a catalytically active fragment thereof, may be introduced into the cellular system as a nucleic acid sequence encoding the site-specific nuclease or the catalytically active fragment thereof, wherein the nucleic acid sequence is part of at least one vector, or wherein the at least one site-specific nuclease or the catalytically active fragment thereof, is introduced into the cellular system as at least one amino acid sequence. In one embodiment, the at least one site-specific nuclease may be introduced as translatable RNA. In yet a further embodiment, the at least one site-specific nuclease may be introduced as part of a complex together with at least one further biomolecule, for example, a gRNA, the gRNA optionally being associated with a RT comprising or being associated with the at least one nucleic acid sequence of interest to be introduced into the cellular system.

Any suitable delivery method to introduce at least one biomolecule into a cell or cellular system can be applied, depending on the cell or cellular system of interest. The term “introduction” as used herein thus implies a functional transport of a biomolecule or genetic construct (DNA, RNA, single- or double-stranded, protein, comprising natural and/or synthetic components, or a mixture thereof) into at least one cell or cellular system, which allows the transcription and/or translation and/or the catalytic activity and/or binding activity, including the binding of a nucleic acid molecule to another nucleic acid molecule, including DNA or RNA, or the binding of a protein to a target structure within the at least one cell or cellular system, and/or the catalytic activity of an enzyme such introduced, optionally after transcription and/or translation. Where pertinent, a functional integration of a genetic construct may take place in a certain cellular compartment of the at least one cell, including the nucleus, the cytosol, the mitochondrium, the chloroplast, the vacuole, the membrane, the cell wall and the like. Consequently, the term “functional integration”—in contrast to the term implies that the molecular complex of interest is introduced into the at least one cell by any means of transformation, transfection or transduction by biological means, including Agrobacterium transformation, or physical means, including particle bombardment, as well as the subsequent step, wherein the molecular complex exerts its effect within or onto the at least one cell or cellular system in which it was introduced. Depending on the nature of the genetic construct or biomolecule to be introduced, said effect naturally can vary and including, alone or in combination, inter alia, the transcription of a DNA encoded by the genetic construct to a RNA, the translation of an RNA to an amino acid sequence, the activity of an RNA molecule within a cell, comprising the activity of a guide RNA, a crRNA, a tracrRNA, or an miRNA or an siRNA for use in RNA interference, and/or a binding activity, including the binding of a nucleic acid molecule to another nucleic acid molecule, including DNA or RNA, or the binding of a protein to a target structure within the at least one cell, or including the integration of a sequence delivered via a vector or a genetic construct, either transiently or in a stable way. Said effect can also comprise the catalytic activity of an amino acid sequence representing an enzyme or a catalytically active portion thereof within the at least one cell and the like. Said effect achieved after functional integration of the molecular complex according to the present disclosure can depend on the presence of regulatory sequences or localization sequences which are comprised by the genetic construct of interest as it is known to the person skilled in the art.

Therefore, a variety of suitable delivery techniques may be suitable according to the methods of the present invention for introducing genetic material into a plant cell or a cellular system derived from a plant cell, the delivery methods being known to the skilled person., e.g., by choosing direct delivery techniques ranging from polyethylene glycol (PEG) treatment of protoplasts (Potrykus et al. 1985), procedures like electroporation (D'Halluin et al., 1992), microinjection (Neuhaus et al., 1987), silicon carbide fiber whisker technology (Kaeppler et al., 1992), viral vector mediated approaches (Gelvin, Nature Biotechnology 23, “Viral-mediated plant transformation gets a boost”, 684-685 (2005)) and particle bombardment (see e.g. Sood et al., 2011, Biologia Plantarum, 55, 1-15).

Despite transformation methods based on biological approaches, like Agrobacterium transformation or viral vector mediated plant transformation, and methods based on physical delivery methods, like particle bombardment or microinjection, have evolved as prominent techniques for introducing genetic material and other biomolecules, including naturally occurring and synthetic biomolecules, or a mixture thereof, into a plant cell or tissue of interest. Helenius et al. (“Gene delivery into intact plants using the Helios™ Gene Gun”, Plant Molecular Biology Reporter, 2000, 18 (3):287-288) discloses a particle bombardment as physical method for introducing material into a plant cell. Currently, there thus exists a variety of plant transformation methods to introduce genetic material in the form of a genetic construct into a plant cell of interest, comprising biological and physical means known to the skilled person on the field of plant biotechnology and which can be applied to introduce at least one gene encoding at least one wall-associated kinase into at least one cell of at least one of a plant cell, tissue, organ, or whole plant. Notably, said delivery methods for transformation and transfection can be applied to introduce the tools of the present invention simultaneously. A common biological means is transformation with Agrobacterium spp. which has been used for decades for a variety of different plant materials. According to the nature of the present invention inter alia relying on a (partially) inactivated Pol theta enzyme, Agrobacterium mediated approaches may also result in a transient introduction of the relevant sequence inserted using Agrobacterium as delivery tool, as T-DNA integration will be hampered.

Viral vector mediated plant transformation represents a further strategy for introducing genetic material into a cell of interest. Physical means finding application in plant biology are particle bombardment, also named biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alia transfection with calcium phosphate, transfection using liposomes, e.g., cationic liposomes, or transfection with cationic polymers, including DEAD-dextran or polyethylenimine, or combinations thereof. Said delivery methods and delivery vehicles or cargos thus inherently differ from delivery tools as used for other eukaryotic cells, including animal and mammalian cells and every delivery method has to be specifically fine-tuned and optimized so that a construct of interest for introducing and/or modifying at least one gene encoding at least one wall-associated kinase in the at least one plant cell, tissue, organ, or whole plant; and/or can be introduced into a specific compartment of a target cell of interest in a fully functional and active way. The above delivery techniques, alone or in combination, can be used for in vivo (in planta) or in vitro approaches. According to the various embodiments of the present invention, different delivery techniques may be combined with each other, for example, using a chemical transfection for the at least one site-specific nuclease, or a mRNA or DNA encoding the same, and optionally further molecules, for example, a gRNA, whereas this is combined with the transient provision of the (partial) inactivation(s) using an Agrobacterium based technique.

In one embodiment according to the various aspects of the present invention, the at least one nucleic acid sequence of interest to be introduced into a cellular system may be selected from the group consisting of: a transgene, a modified endogenous gene, a synthetic sequence, an intronic sequence, a coding sequence or a regulatory sequence.

In another embodiment according to the various aspects of the present invention, there is provided a method, wherein the at least one nucleic acid sequence of interest to be introduced into a cellular system is a transgene, wherein the transgene comprises a nucleic acid sequence encoding a gene of a genome of an organism of interest, or at least a part of said gene.

In one embodiment, a regulatory sequence according to the present invention may be a promoter sequence, wherein the editing or mutation or modulation of the promoter comprises replacing the promoter, or promoter fragment with a different promoter (also referred to as replacement promoter) or promoter fragment (also referred to as replacement promoter fragment), wherein the promoter replacement results in any one of the following or any one combination of the following: an increased promoter activity, an increased promoter tissue specificity, a decreased promoter activity, a decreased promoter tissue specificity, a new promoter activity, an inducible promoter activity, an extended window of gene expression, a modification of the timing or developmental progress of gene expression in the same cell layer or other cell layer, for example, extending the timing of gene expression in the tapetum of anthers, a mutation of DNA binding elements and/or a deletion or addition of DNA binding elements. The promoter (or promoter fragment) to be modified can be a promoter (or promoter fragment) that is endogenous, heterologous, artificial, pre-existing, or transgenic to the cell that is being edited. The replacement promoter or fragment thereof can be a promoter or fragment thereof that is endogenous, heterologous, artificial, pre-existing, or transgenic to the cell that is being edited. Any other regulatory sequence according to the present disclosure may be modified as detailed for a promoter or promoter fragment above.

In a preferred embodiment and in case of plant genomes to be modified, it is highly desirable that the modification as mediated by the methods of the present invention does not result in a genetically modified, transgenic organism by integrating foreign DNA into the parent genome in an imprecise way, as environmental, regulatory and political issues have to be concerned. Therefore, the embodiments according to the present invention providing methods for modifying a genetic material of interest in a cellular system in a transient way are particularly suitable for providing a cellular system comprising a modification at a predetermined location without inserting foreign DNA and thus without providing a cell or organism regarded as genetically modified organism, as all tools necessary to perform the methods of the present invention can be provided to the cellular system in a transient way in active form.

In certain embodiments, it may be suitable to introduce a sequence encoding the at least one site-specific nuclease as knock-in, and/or to provide a (partial) inactivation of the sequence encoding the Pol theta, and/or to provide a (partial) inactivation of the at least one further NHEJ pathway repair enzyme in a donor genome or genetic material to be modified in a stable way to provide a genetic background assisting in performing the methods of the present invention. In these embodiments, it can be favorable to restore the integrity of the donor genome after a modification has been performed according to the methods of the present invention so that the stable mutation and/or knock-in and/or knock-out introduced before GE is then again restored by crossing and/or selection or other suitable technical means of molecular biology, cell culture, or haploidization.

As the methods of the present invention comprise the introduction of more than one biomolecule and/or the additional (partial) inactivation of at least one Pol theta enzyme and of at least one further NHEJ pathway enzyme, the methods may be performed in a fully transient way. In other embodiments, the methods may be performed by a combination of stable and transient approaches. In yet a further embodiment, the methods may also be performed by stably introducing suitable delivery tools to a cell or cellular system of interest.

In a further embodiment according to the various aspects of the present invention, a further modification at a predetermined location is introduced resulting in the introduction of a selection marker into the genetic material of the cellular system.

Edited plants can be easily identified and separated from non-edited plants, when they are co-edited with selectable markers. Based on specific resistance or visual markers, screenings can be performed. Any endogenous gene which could be modified in a convenient way which confers either a resistance or a phenotypic marker (e.g. shape, color, fluorescence etc.) could be used. Phenotypic examples might be e.g. glossy genes, golden, zebra7/lemonwhite1, tiedyed, nitrate reductase family members (for corn and sugar beet) and the like (see e.g. the disclosure of U.S. 62/502,418 which is incorporated by reference in its entirety).

Non-limiting examples of resistance and or phenotypic marker include herbicide resistance/tolerance, wherein the herbicide resistance/tolerance is selected from the group consisting of resistance/tolerance to EPSPS-inhibitors, including glyphosate, resistance/tolerance to glutamine synthesis inhibitors, including glufosinate, resistance/tolerance to ALS- or AHAS-inhibitors, including imidazoline or sulfonylurea, resistance/tolerance to ACCase inhibitors, including aryloxyphenoxypropionate (FOP), resistance/tolerance to carotenoid biosynthesis inhibitors, including inhibitors of carotenoid biosynthesis at the phytoene desaturase step, inhibitors of 4-hydroxyphenyl-pyruvate-dioxygenase (HPPD), or inhibitors of other carotenoid biosynthesis targets, resistance/tolerance to cellulose inhibitors, resistance/tolerance to lipid synthesis inhibitors, resistance/tolerance to long-chain fatty acid inhibitors, resistance/tolerance to microtubule assembly inhibitors, resistance/tolerance to photosystem I electron diverters, resistance/tolerance to photosystem II inhibitors, including carbamate, triazines and triazinones, resistance/tolerance to PPO-inhibitors and resistance/tolerance to synthetic auxins, including dicamba (2,4-D, i.e., 2,4-dichlorophenoxyacetic acid).

In one embodiment according to the various aspects of the present invention, the at least one nucleic acid sequence of interest to be introduced into a cellular system may be selected from the group consisting of: a transgene, a cisgene, a modified endogenous gene, a synthetic sequence, an intronic sequence, a coding sequence or a regulatory sequence.

In still another embodiment according to the various aspects of the present invention, the at least one nucleic acid sequence of interest to be introduced into a cellular system at a predetermined location may be a transgene, or part of a transgene, or a cisgene, or part of a cisgene, of an organism of interest, wherein the transgene, the cisgene or part thereof is selected from the group consisting of a gene encoding tolerance to abiotic stress, including drought stress, osmotic stress, heat stress, chilling stress, cold stress including frost, oxidative stress, heavy metal stress, nitrogen deficiency, phosphate deficiency, salt stress or waterlogging, herbicide resistance, including resistance to glyphosate, glufosinate/phosphinotricin, hygromycin, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, bolting resistance, flowering time, shattering resistance, seed color, endosperm composition, or nutritional content.

In one embodiment according to the various aspects of the present invention, the at least one nucleic acid sequence of interest to be introduced into a cellular system at a predetermined location may be at least part of a modified endogenous gene of an organism of interest, wherein the modified endogenous gene comprises at least one deletion, insertion and/or substitution of at least one nucleotide in comparison to the nucleic acid sequence of the unmodified (wild-type) endogenous gene.

In another embodiment according to the various aspects of the present invention, the at least one nucleic acid sequence of interest to be introduced into a cellular system at a predetermined location may be at least part of a modified endogenous gene of an organism of interest, wherein the modified endogenous gene comprises at least one of a truncation, duplication, substitution and/or deletion of at least one nucleic acid position encoding a domain of the modified endogenous gene.

In yet another embodiment according to the various aspects of the present invention, the at least one nucleic acid sequence of interest to be introduced into a cellular system at a predetermined location may be at least part of a regulatory sequence, wherein the regulatory sequence comprises at least one of a core promoter sequence, a proximal promoter sequence, a cis acting element, a trans acting element, a locus control sequences, an insulator sequence, a silencer sequence, an enhancer sequence, a terminator sequence, a conserved motif of a regulatory element like TATA box and/or any combination thereof.

One embodiment of the above methods according to the present invention is a method for modifying a eukaryotic cell, preferably at least one plant cell, or a cellular system comprising the genetic material, or part of the genetic material thereof, in a targeted way to provide a genetically modified, preferably non-transgenic plant, wherein the method may inter alia be a method for trait development. For example, a highly site-specific substitution of 1, 2, 3 or more nucleotides in the coding sequence of a plant gene can be introduced so as to produce substitutions of one or more amino acids that will confer tolerance to at least one herbicide such as glyphosate, glufosinate, Dicamba or an acetolactate synthase (ALS) inhibiting herbicide. Furthermore, in another embodiment, substitutions of one or more amino acids in the coding sequence of a nucleotide binding site-leucine-rich repeat (NBS-LRR) plant gene that will alter the pathogen recognition spectrum of the protein to optimize the plant's disease resistance. In yet a further embodiment, a small enhancer sequence or transcription factor binding site can be modified in an endogenous promoter of a plant gene or can be introduced into the promoter of a plant gene so as to alter the expression profile or strength of the plant gene regulated by the promoter. The expression profile can be altered through various modifications, introductions or deletions in other regions, such as introns, 3′ untranslated regions, cis- or trans-enhancer sequences. In yet a further embodiment, the genome of a plant cell, preferably a meristematic plant cell, can be modified in a way so that the plant resulting from the modified meristematic cell, can produce a chemical substance or compound of agronomic or pharmaceutical interest, for example insulin or insulin analoga, antibodies, a protein with an enzymatic function of interest, or any other pharmaceutically relevant compound suitable as medicament, as dietary supplement, or as health care product.

Non limiting examples of traits that can be introduced by the method according to this embodiment are resistance or tolerance to insect pests, such as to rootworms, stem borers, cutworms, beetles, aphids, leafhoppers, weevils, mites and stinkbugs. These could be made by modification of plant genes, for example, to increase the inherent resistance of a plant to insect pests or to reduce its attractiveness to said pests. Other traits can be resistance or tolerance to nematodes, bacterial, fungal or viral pathogens or their vectors. Still other traits could be more efficient nutrient use, such as enhanced nitrogen use, improvements or introductions of efficiency in nitrogen fixation, enhanced photosynthetic efficiency, such as conversion of C3 plants to C4. Yet other traits could be enhanced tolerance to abiotic stressors such as temperature, water supply, salinity, pH, tolerance for extremes in sunlight exposure. Additional traits can be characteristics related to taste, appearance, nutrient or vitamin profiles of edible or feedable portions of the plant, or can be related to the storage longevity or quality of these portions. Finally, traits can be related to agronomic qualities such resistance to lodging, shattering, flowering time, ripening, emergence, harvesting, plant structure, vigor, size, yield, and other characteristics.

In one embodiment according to the various aspects of the present invention, the at least one site-specific nuclease may comprise a zinc-finger nuclease, a transcription activator-like effector nuclease, a CRISPR/Cas system, including a CRISPR/Cas9 system, a CRISPR/Cpf1 system, a CRISPR/CasX system, a CRISPR/CasY system, an engineered homing endonuclease, and a meganuclease, and/or any combination, variant, or catalytically active fragment thereof.

A CRISPR system in its natural environment describes a molecular complex comprising at least one small and individual non-coding RNA in combination with a Cas nuclease or another CRISPR nuclease like a Cpf1 nuclease (Zetsche et al., 2015, supra) which can produce a specific DNA double-stranded break. Presently, CRISPR systems are categorized into 2 classes comprising five types of CRISPR systems, the type II system, for instance, using Cas9 as effector and the type V system using Cpf1 as effector molecule (Makarova et al., Nature Rev. Microbiol., 2015). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems which have been characterized so far. Firstly, the invading DNA, also known as a spacer, is integrated between two adjacent repeat regions at the proximal end of the CRISPR locus. Type II CRISPR systems, for example, can code for a Cas9 nuclease as key enzyme for the interference step, which system contains both a crRNA and also a trans-activating RNA (tracrRNA) as the guide motif. These hybridize and form double-stranded (ds) RNA regions which are recognized by RNAseIII and can be cleaved in order to form mature crRNAs. These then in turn associate with the Cas molecule in order to direct the nuclease specifically to the target nucleic acid region. Recombinant gRNA molecules can comprise both the variable DNA recognition region and also the Cas interaction region and thus can be specifically designed, independently of the specific target nucleic acid and the desired Cas nuclease. As a further safety mechanism, PAMs (protospacer adjacent motifs) must be present in the target nucleic acid region; these are DNA sequences which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337: 816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973 A1). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

Presently, for example, Type II systems relying on Cas9, or a variant or any chimeric form thereof, as endonuclease have been modified for genome engineering. Synthetic CRISPR systems consisting of two components, a guide RNA (gRNA) also called single guide RNA (sgRNA) and a non-specific CRISPR-associated endonuclease can be used to generate knock-out cells or animals by co-expressing a gRNA specific to the gene to be targeted and capable of association with the endonuclease Cas9. Notably, the gRNA is an artificial molecule comprising one domain interacting with the Cas or any other CRISPR effector protein or a variant or catalytically active fragment thereof and another domain interacting with the target nucleic acid of interest and thus representing a synthetic fusion of crRNA and tracrRNA (as “single guide RNA” (sgRNA) or simply “gRNA”). The genomic target can be any ˜20 nucleotide DNA sequence, provided that the target is present immediately upstream of a PAM sequence. The PAM sequence is of outstanding importance for target binding and the exact sequence is dependent upon the species of Cas9 and, for example, reads 5′ NGG 3′ or 5′ NAG 3′ (Standard IUPAC nucleotide code) (Jinek et al., Science 2012, supra) for a Streptococcus pyogenes derived Cas9. The PAM sequence for Cas9 from Staphylococcus aureus is NNGRRT or NNGRR(N). Many further variant CRISPR/Cas9 systems are known, including inter alia, Neisseria meningitidis Cas9 cleaving the PAM sequence NNNNGATT. A Streptococcus thermophilus Cas9 cleaving the PAM sequence NNAGAAW. Using modified Cas nucleases, targeted single-strand breaks can be introduced into a target sequence of interest. By the combined use of such a Cas nickase with different recombinant gRNAs highly site specific DNA double-strand breaks can be introduced using a double nicking system. Using one or more gRNAs can further increase the overall specificity and reduce off-target effects.

Once expressed, the Cas9 protein and the gRNA form a ribonucleoprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex will bind any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA will continue to anneal to the target DNA in a 3′ to 5′ direction (relative to the polarity of the gRNA).

CRISPR/Cas, e.g. CRISPR/Cas9, and likewise CRISPR/Cpf1 or CRISPR/CasX or CRISPR/CasY and other CRISPR systems are highly specific when gRNAs are designed correctly, but especially specificity is still a major concern, particularly for clinical uses or targeted plant GE based on the CRISPR technology. The specificity of the CRISPR system is determined in large part by how specific the gRNA targeting sequence is for the genomic target compared to the rest of the genome. Therefore, the methods according to the present invention when combined with the use of at least one CRISPR nuclease as site-specific nuclease and further combined with the use of a suitable CRISPR nucleic acid can provide a significantly more predictable outcome of GE. Whereas the CRISPR complex can mediate a highly precise cut of a genome or genetic material of a cell or cellular system at a specific site, the methods presented herein provide an additional control mechanism guaranteeing a programmable and predictable repair mechanism.

According to the various embodiments of the present invention, the above disclosure with respect to covalent and non-covalent association or attachment also applies for CRISPR nucleic acids sequences, which may comprise more than one portion, for example, a crRNA and a tracrRNA portion, which may be associated with each other as detailed above. In one embodiment, a RT nucleic acid sequence of the present invention may be placed within a CRISPR nucleic acid sequence of interest to form a hybrid nucleic acid sequence according to the present invention, which hybrid may be formed by covalent and non-covalent association.

In yet a further embodiment according to the various aspects of the present invention, the one or more nucleic acid sequence(s) flanking the at least one nucleic acid sequence of interest at the predetermined location may have at least 85%-100% complementary to the one or more nucleic acid sequence(s) adjacent to the predetermined location, upstream and/or downstream from the predetermined location, over the entire length of the respective adjacent region(s). Notably, a lower degree of homology or complementarity of the at least one flanking region may be used, e.g. at least 70%, at least 75%, at least 80%, at least 81%, at least 82%, at least 83%, or at least 84% homology/complementarity to at least one adjacent region in the genetic material of interest. For high precision GE relying on HDR template, i.e., a RT as disclosed herein, more than 95% homology/complementarity are favorable to achieve a highly targeted repair event. As shown in Rubnitz et al., Mol. Cell Biol., 1984, 4(11), 2253-2258, also very low sequence homology might suffice to obtain a homologous recombination. As it is known to the skilled person, the degree of complementarity will depend on the genetic material to be modified, the nature of the planned edit, the complexity and size of a genome, the number of potential off-target sites, the genetic background and the environment within a cell or cellular system to be modified.

In yet a further embodiment according to the various aspects of the present invention, the genetic material of the cellular system may be selected from the group consisting of a protoplast, a viral genome transferred in a recombinant host cell, a eukaryotic or prokaryotic cell, tissue, or organ, and a eukaryotic or prokaryotic organism, preferably a eukaryotic organism. Even though prokaryotic organism may not themselves comprise Pol theta or other enzymes of a NHEJ pathway, a prokaryotic genome, or parts thereof, may still represent a genetic material according to the present invention, for example, in case all or part of a prokaryotic genome is transferred into a eukaryotic host cell as cellular system, i.e., a prokaryotic donor genome may be modified in the context of a eukaryotic host molecular system.

In one embodiment according to the various aspects of the present invention, the genetic material of the cellular system may be selected from a eukaryotic cell, wherein the eukaryotic cell is preferably a plant cell.

In certain embodiments, the methods of the present invention can thus be suitable for use in a method of treatment a disease, wherein the disease is characterized by at least one genomic mutation and the artificial molecular complex is configured to target and repair the at least one genomic mutation resulting in a disease phenotype. There is thus provided a method of treating a disease using the artificial molecular complex according to any one of the preceding claims, wherein the disease is characterized by at least one genomic mutation and the artificial molecular complex is configured to target and repair the at least one genomic mutation. The therapeutic method of treatment may comprise gene or genome editing, or gene therapy.

In certain embodiments, the genetic material to be modified from at least one eukaryotic cell may be a meristematic plant cell, and the plant cell, after (partial) inactivation of Pol theta and at least one further repair enzyme of a NHEJ pathway and introduction of GE tools according to the present invention is further cultivated under suitable conditions until the developmental stage of maturity of the inflorescence is achieved to obtain a plant or plant material comprising a modification of interest mediated by the at least one molecular complex according to the present invention. Several protocols are, for example, available to the skilled person for producing germinable and viable pollen from in vitro cultured maize tassels, for example in Pareddy D R et al. (1992) Maturation of maize pollen in vitro. Plant Cell Rep 11 (10):535-539. doi:10.1007/BF00236273, Stapleton A E et al. (1992) Immature maize spikelets develop and produce pollen in culture. Plant. Cell Rep., 11 (5-6):248-252, or Pareddy D R et al. (1989) Production of normal, germinable and viable pollen from in vitro-cultured maize tassels, Theor. Appl. Genet. 77 (4):521-526. Those protocols are inter alia based on excision of the tassel, surface sterilization and culture in a media with kinetin to promote tassel growth and maturation. After the spikelets are formed, a continuous harvest of anthers can be performed. After extrusion, anthers will be desiccated until the pollen comes out. Alternatively, anthers can be dissected and the pollen is shed in liquid medium that is subsequently used to pollinate ears.

“Maturity of the inflorescence” as used herein refers to the state, when the immature inflorescence of a plant comprising at least one meristematic cell has reached a developmental stage, when a mature inflorescence, i.e. a staminate inflorescence (male) or a pistillate inflorescence (female), is achieved and thus a gamete of the pollen (male) or of the ovule (female) or both is present. Said stage of the reproductive phase of a plant is especially important, as obtained plant material can directly be used for pollination of a further plant or for fertilization with the pollen of another plant.

By generating cells or cellular systems that harbor a mutation in Pol θ together with a mutation in an enzyme essential for NHEJ, for example, Ku70, Ku80, or Ligase IV (LigIV) and other targets disclosed herein, it is possible to produce cells or cellular systems having complete dominance of the HDR pathway with no random (or untargeted) integration of foreign DNA. Performing gene targeting experiments in said cells or cellular systems, and particularly in plant cells or cellular systems, harboring the double mutations has several benefits. First, by inhibiting the NHEJ pathway, this prevents SSN-induced DSBs from being repaired by this pathway so they remain open and available for HDR. Second, by inhibiting Pol theta, there is no random integration of the RT or any of the transgene cassettes (e.g., SSN cassette, fluorescent reporters, plasmid backbone, etc.) to interfere with the screening of cell lines or organisms for gene targeting. The present invention provides methods particularly suitable for plant GE and taking into consideration the complexity of plant genomes to avoid a significant loss of viability of these at least double mutant or double impaired cells with respect to the NHEJ enzymes to provide cellular systems comprising a (partially) inactivated Pol theta and at least one further enzyme having an increased HDR rate when GE is performed. Therefore, the methods disclosed herein provide an ideal environment for gene targeting, in which the dominant mechanism available to repair DSBs is by HDR.

Another strategy and preferred embodiments described herein are the transient (partial) inhibition of Pol theta and the NHEJ pathway in cells or cellular systems, while simultaneously delivering an SSN and RT. This can be done with interfering RNA directed against Pol theta and either Ku70, Ku80, ligase IV, or another essential NHEJ enzyme as disclosed herein.

By protein interference with these enzymes such as, for example, by delivering adenovirus 4 E1B55K and E4orf6 proteins which inhibit ligase IV; by delivering small chemical inhibitors of these enzymes such as, for example, SCR7, W7, Vanillin, NU7026, NU7441 (Arras & Fraser, 2016, PLOS ONE 11(9): e0163049) which inhibits ligase IV, DNA PKcs, Ku cofactor synthesis; or by any combination of these and the mutation methods. Other chemical or synthetic, and/or biological inhibitors of any enzyme of a NHEJ pathway disclosed herein may be used which inhibitor can be administered to a cell or cellular system in a dose non-toxic to the cell or cellular system to guarantee viability of the cell or cellular system, wherein the dose is sufficient to at least partially inhibit the activity of Pol theta and at least one further enzyme of a NHEJ pathway, preferably in a transient way.

As it is known to the skilled person and as defined above, RNAi relies on the action of small RNAs, which may be selected from a micro RNA (miRNA), a small interfering RNA (siRNA), or a Piwi-interacting RNA (piRNA), comprising naturally and/or non-naturally occurring (synthetic) ribonucleotides, wherein synthetic nucleotide, e.g. comprising a phosphorothioate backbone, might be suitable to enhance stability of the usually easily degradable RNA molecule. SiRNAs of ˜21 nt have been reported to play a crucial role in RNA silencing, a term referring to post-transcriptional gene silencing in plants, quelling in fungi and RNAi animals. The mechanism of siRNA biogenesis and function are thought to be highly conserved in almost all the eukaryotes including plants and animals, in which siRNAs are produced from double-stranded RNA (dsRNA) by an RNase III termed Dicer in animal cells or DCL (Dicer-like) in plants, and then incorporated into a RNA-induced silencing complex (RISC), in which siRNAs play a guiding role in sequence-specific cleavage of target mRNAs. Moreover, in some organisms, such as Caenorhabditis elegans, Drosophila and plants, the siRNA signal is found to spread along the mRNA target, which results in the production of secondary siRNAs and the induction of transitive RNA silencing (see Lu et al., Nucleic. Acids Res., 2004, 32(21):e171).

In other embodiments, an RNA interference (RNAi) mechanism may thus be used to achieve a transient inhibition of activity of at least one Pol theta and at least one further NHEJ enzyme. The interfering RNA can trigger silencing of the mRNAs for relevant effector enzymes of at least one NHEJ pathway. It can be delivered as double-stranded RNA, as single-stranded antisense RNA, in hairpin DNA expression cassettes, or as chimeric poly-sgRNA/siRNA sequences which generate multiple sgRNA-Cas9 RNP complexes upon the Dicer-mediated digestion of the siRNA parts, leading to more efficient disruption of the target gene in cells (Ha J. S. et al., Journal of Controlled Release 250 (2017) 27-35).

The (partial) transient inhibition according to the various embodiments disclosed herein can inhibit or inactivate a Pol theta and at least one further NHEJ enzyme in a different degree, for example, the activity of a Pol theta enzyme may be fully inactivated, whereas the activity of at least one further NHEJ pathway enzyme may be partially inactivated and vice versa.

According to the various aspects and embodiments of the present invention, it is contemplated that a transient (partial) inactivation can comprise a combination of at least one of a RNAi silencing mechanism acting on the RNA level, and/or a chemical/synthetic or biological inhibitor acting on the RNA or protein level of an enzyme to be inactivated, and/or an inhibitor acting, for example, in trans to regulate transcription of a Pol theta and at least one further NHEJ pathway enzyme.

In a further embodiment according to the various aspects of the present invention, there is provided a method, wherein the eukaryotic organism may be a plant, or a part of a plant. In yet a further embodiment according to the various aspects of the present invention, the part of the plant may be selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

In one embodiment according to the various aspects of the present invention, the genetic material of the cellular system may be, or may originate from, a plant species selected from the group consisting of: Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Secale cereale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Astragalus sinicus, Lotus japonicas, Torenia fournieri, Allium cepa, Allium fistulosum, Allium sativum, and Allium tuberosum. Based on the disclosure provided herein, the methods of the present invention can easily be transferred and can be used for the modification of the genetic material obtained from other plants or plant species.

In a further aspect, there is provided a method for producing a cellular system, preferably a cellular system as defined herein above, comprising the following steps: (a) providing a cellular system or a genetic material of a cellular system comprising a functional Polymerase theta enzyme, or the sequence encoding the same, and one or more further functional DNA repair enzyme(s), or the sequence(s) encoding the same, of the NHEJ pathway; (b) inactivating or partially inactivating the Polymerase theta enzyme, or the sequence encoding the same, and inactivating or partially inactivating one or more further DNA repair enzyme(s), or the sequence(s) encoding the same, wherein the inactivation or partial inactivation takes place simultaneously or subsequently, preferably in a transient manner; (c) optionally, introducing the genetic material into a cellular system, (d) obtaining a cellular system comprising a functionally inactivated or partially inactivated Polymerase theta enzyme and one or more further functionally inactivated or partially inactivated DNA repair enzyme(s). This aspect may be particularly suitable to provide a cellular system and/or a genetic material to be further modified by any method of GE to provide a cell or system having an at least impaired endogenous NHEJ pathway, at least for a transient period of time, for example, to test for optimum GE conditions.

In one embodiment, the inactivation or partial inactivation may be a stable inactivation, or the inactivation or partial inactivation may be a transient inactivation, preferably a transient inactivation or partial inactivation based on a gene silencing machinery, including RNAi, or a chemical inhibitor, or any combination thereof. Preferably all alleles of the Polymerase theta enzyme and/or the one or more further DNA repair enzyme(s) of a NHEJ pathway are inactivated or partially inactivated, i.e. a knock-out of the Polymerase theta enzyme and/or the one or more further DNA repair enzyme(s) of a NHEJ pathway is present homozygously.

In a further embodiment according to the various aspects disclosed herein, the modification or inactivation or partial inactivation may comprise a modification of at least one nucleic acid sequence encoding the Polymerase theta enzyme and of at least one nucleic acid sequence encoding one or more further DNA repair enzyme(s) of a NHEJ pathway, wherein the at least one modification may comprise at least one deletion, insertion or substitution of at least one nucleotide in the respective encoding nucleic acid sequence resulting in the alteration of the corresponding amino acid sequence in the encoded enzymes.

In a further embodiment according to the various aspects disclosed herein, the Polymerase theta enzyme and the one or more further DNA repair enzyme of the NHEJ pathway are inactivated or partially inactivated by a gene silencing/inactivation machinery. The embodiment using a gene silencing/inactivation machinery will usually rely on a RNAi machinery and may be particularly suitable for a transient (partial) inactivation to guarantee that the Pol theta and the one or more further DNA repair enzyme of the NHEJ pathway can easily be reactivated to fulfill its natural function in DSB break repair after a targeted GE event has been introduced.

The at least one Polymerase theta enzyme and the one or more further DNA repair enzyme of the NHEJ pathway to be inactivated or partially inactivated according to the aspects disclosed herein directed to at least one cellular system may be selected from the sequences as defined herein above.

In certain embodiments, the gene silencing/inactivation machinery may selected from a system comprising (i) at least one small interfering RNA, selected from a DNA hairpin cassette, or interfering RNA, wherein the interfering RNA may comprise a double-stranded RNA, optionally comprising a hairpin structure, or a single-stranded sense and/or antisense RNA; optionally comprising (ii) a site specific RNA endonuclease, such as C2c2; and optionally comprising (iii) at least one of an adenovirus 4 E1B55K and/or E4orf6 protein, or the sequence encoding the same; and/or optionally comprising (iv) at least one small chemical inhibitor selected from the group consisting of: SCR7, W7, Vanillin, NU7026 and NU7441.

In one embodiment relying on RNAi as transient (partial) inactivation mechanism, first, uniqueness of a RNA inhibitory molecule sequence of interest used as silencer in a genome or genetic material of interest is confirmed. Then sequences about 100 to about 1.000 bp, preferably about 250 to about 500 bp, from the 3′UTR of an mRNA of interest encoding an enzyme to be inhibited are designed. These sequences may be used to be integrated into a hairpin vector or a hairpin construct, or to be used as sense and antisense sequences, to down-regulate expression of a gene on RNA level precisely.

### Delivery Methods:

A variety of suitable transient and stable delivery techniques suitable according to the methods of the present invention for introducing genetic material, biomolecules, including any kind of single-stranded and double-stranded DNA and/or RNA, or amino acids, synthetic or chemical substances, into a eukaryotic cell, preferably a plant cell, or into a cellular system comprising genetic material of interest, are known to the skilled person, and comprise inter alia choosing direct delivery techniques ranging from polyethylene glycol (PEG) treatment of protoplasts (Potrykus et al. 1985), procedures like electroporation (D'Halluin et al., 1992), microinjection (Neuhaus et al., 1987), silicon carbide fiber whisker technology (Kaeppler et al., 1992), viral vector mediated approaches (Gelvin, Nature Biotechnology 23, “Viral-mediated plant transformation gets a boost”, 684-685 (2005)) and particle bombardment (see e.g. Sood et al., 2011, Biologia Plantarum, 55, 1-15). Transient transfection of mammalian cells with PEI is disclosed in Longo et al., Methods Enzymol., 2013, 529:227-240. Protocols for transformation of mammalian cells are disclosed in Methods in Molecular Biology, Nucleic Acids or Proteins, ed. John M. Walker, Springer Protocols.

For plant cells to be modified, despite transformation methods based on biological approaches, like Agrobacterium transformation or viral vector mediated plant transformation, and methods based on physical delivery methods, like particle bombardment or microinjection, have evolved as prominent techniques for introducing genetic material into a plant cell or tissue of interest. Helenius et al. (“Gene delivery into intact plants using the Helios™ Gene Gun”, Plant Molecular Biology Reporter, 2000, 18 (3):287-288) discloses a particle bombardment as physical method for introducing material into a plant cell. Currently, there thus exists a variety of plant transformation methods to introduce genetic material in the form of a genetic construct into a plant cell of interest, comprising biological and physical means known to the skilled person on the field of plant biotechnology and which can be applied to introduce at least one gene encoding at least one wall-associated kinase into at least one cell of at least one of a plant cell, tissue, organ, or whole plant. Notably, said delivery methods for transformation and transfection can be applied to introduce the tools of the present invention simultaneously. A common biological means is transformation with Agrobacterium spp. which has been used for decades for a variety of different plant materials. Viral vector mediated plant transformation represents a further strategy for introducing genetic material into a cell of interest. Physical means finding application in plant biology are particle bombardment, also named biolistic transfection or microparticle-mediated gene transfer, which refers to a physical delivery method for transferring a coated microparticle or nanoparticle comprising a nucleic acid or a genetic construct of interest into a target cell or tissue. Physical introduction means are suitable to introduce nucleic acids, i.e., RNA and/or DNA, and proteins. Likewise, specific transformation or transfection methods exist for specifically introducing a nucleic acid or an amino acid construct of interest into a plant cell, including electroporation, microinjection, nanoparticles, and cell-penetrating peptides (CPPs). Furthermore, chemical-based transfection methods exist to introduce genetic constructs and/or nucleic acids and/or proteins, comprising inter alia transfection with calcium phosphate, transfection using liposomes, e.g., cationic liposomes, or transfection with cationic polymers, including DEAD-dextran or polyethylenimine, or combinations thereof. Said delivery methods and delivery vehicles or cargos thus inherently differ from delivery tools as used for other eukaryotic cells, including animal and mammalian cells and every delivery method has to be specifically fine-tuned and optimized so that a construct of interest for introducing and/or modifying at least one gene encoding at least one wall-associated kinase in the at least one plant cell, tissue, organ, or whole plant; and/or can be introduced into a specific compartment of a target cell or cellular system of interest in a fully functional and active way. The above delivery techniques, alone or in combination, can be used for in vivo (including in planta) or in vitro approaches. In particular for embodiments relying on the transient introduction strategies, RNA-based silencing molecules or chemical, synthetic, or biological inhibitors of at least one of a Pol theta and/or a further enzyme of a NHEJ pathway can, for example, be introduced together with, before, or subsequently to the transformation and/or transfection of relevant tools for GE.

Depending on the nature of the molecule introduced, e.g., a rather stable vector in comparison to a rather unstable RNA molecule, different time schemes of transformation/transfection should be chosen to guarantee that the (partial) inactivation of Pol theta and at least one further NHEJ pathway enzyme is available exactly at the time point when the GE tools are available or provided to one and the same cell. RNAi-based down-regulation of a target may thus need some time to become active. Likewise, in case a molecule is introduced as transcribable/translatable (plasmid) vector, it may take some time until the tools can be provided in their active form and are available in the right compartment within a cell or cellular system of interest. To be able to provide highly active molecules to a cellular system of interest, in certain embodiments it may thus be preferred to provide pre-assembled and function molecular complexes comprising at least one site-specific nuclease, optionally at least one gRNA (for CRISPR nucleases), and further providing a nucleic acid sequence of interest, preferably flanked by at least one homology region in the form of a repair template, to be able to provide a fully functional GE complex to a cell or cellular system exactly synchronized with (partial) inactivation of Pol theta and at least one further NHEJ pathway enzyme.

In particular with respect to embodiments directed to the provision of methods for providing a modified genetic material of a plant cell, or for providing a whole plant comprising modified genetic material, transient methods may be preferable due to legal and regulatory concerns.

In one aspect according to the present invention, there is thus provided a plant cell, tissue, organ, whole plant or plant material, or a derivative or a progeny thereof, obtainable by a method as disclosed herein, wherein the methods optionally comprise a further step of breeding or crossing.

The present invention is further described with reference to the following non-limiting examples.

### EXAMPLES

### Example 1: Generation of Double Mutants in Arabidopsis thaliana

To test whether double mutants of Pol θ (PolQ) and at least one mutant from the group of Ku70, Ku80 or LigIV are viable and could be used for further studies, the following Arabidopsis T-DNA insertion mutant lines were commercially obtained: NASC-IDs N698253, N667884, N656936, N677892 and N656431 (see Table 1 below).

TABLE 1
Overview of the tested mutant lines

| | | Line | | |
|---|---|---|---|---|
| Gene notation | AGI-ID | notation | T-DNA | NASC-ID |
| Pol θ, TEB | At4g32700 | teb-2 | SALK_035610C | N698253 |
| | | teb-5 | SALK_018851C | N667884 |
| KU70 | At1g16970 | ku70 | SALK_123114C | N656936 |
| KU80 | At1g48050 | ku80 | SALK_112921C | N677892 |
| LIGIV | At5g57160 | ligIV | SALK_044027C | N656431 |

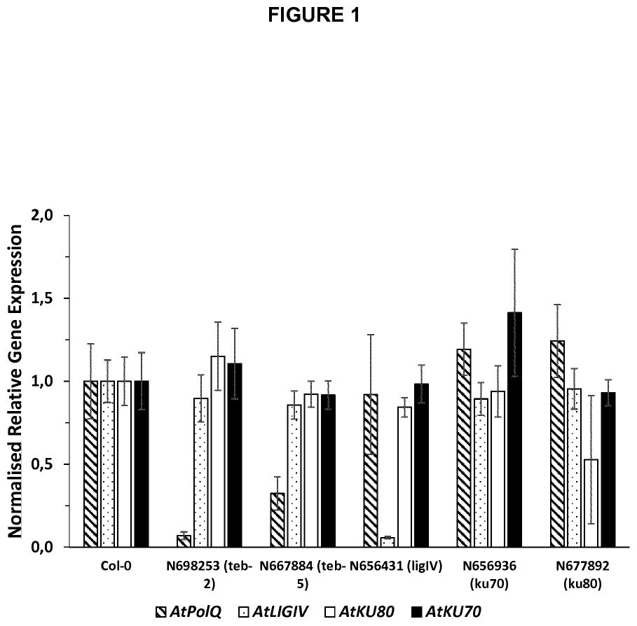

T-DNA insertion and expression of disrupted genes were determined by PCR/qRT-PCR (FIG. 1). Next, all mutant lines were grown until flowering, and the two PolQ (At4g32700) mutants (teb-2 and teb-5) were each crossed with the Ku70 (At1g16970), Ku80 (At1g48050) or LigIV (At5g57160) mutants to obtain the respective double mutants. Importantly, all crossings resulted in viable seeds which were harvested and propagated to F2. F2 plants were characterized by PCR for T-DNA insertion into both alleles of PolQ, Ku70, Ku80 and LigIV, respectively. For 5 of the 6 crossings, plants with T-DNA insertions into both alleles of both genes were identified. For the teb-2×ku70 crossing, no homozygous double mutants were identified (Table 2). The obtained rates were significantly lower than expected, indicating that especially the Ku-double mutants have some fertility problems. All double mutants showed no severe growth phenotypes, even though some plants showed reduced growth. F3 seeds were harvested from these plants (Table 3). None of the identified double mutants showed severe fertility defects. It was thus possible to obtain enough seeds for all double mutants for subsequent floral dip experiments.

TABLE 2
Overview of F3 generations obtained from double mutant lines.

| Double mutant lines | Generation |
|---|---|
| teb-2 × ligIV | F3 |
| teb-5 × ligIV | F3 |
| teb-5 × ku70 | F3 |
| teb-2 × ku70 | No homozygous plant |
| teb-2 × ku80 | F3 |
| teb-2 × ku80 | F3 |

### Example 2: Generation of Gene Targeting Construct for Testing Gene Targeting Frequencies

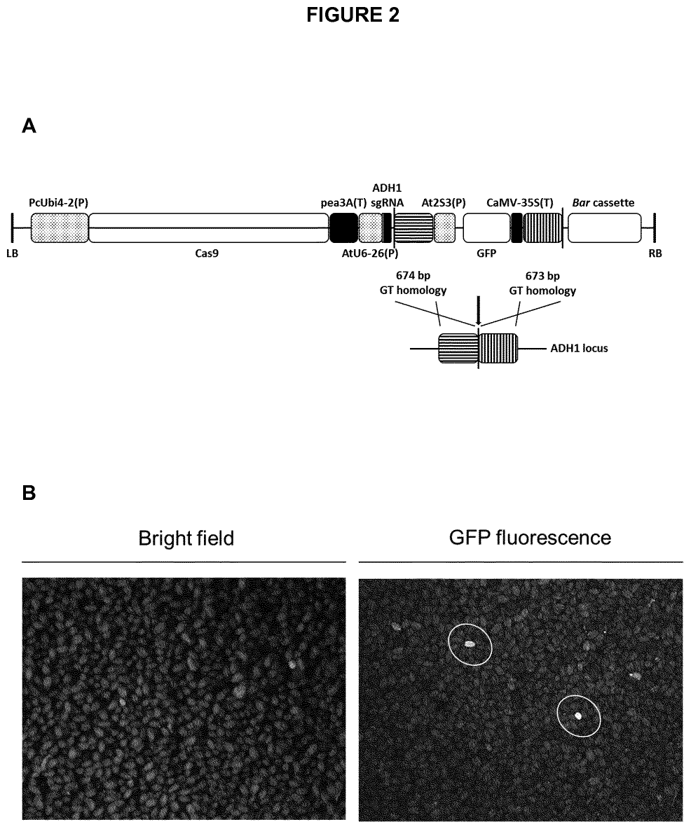

For determination of gene targeting fequencies, a construct based on the gene targeting construct “pFF15”, described by Shiml, Fauser and Puchta (2014), was designed targeting the ADH1 (alcohol dehydrogenase 1) locus (FIG. 2A; SEQ ID NO: 82). The construct contains a Bar selection marker to allow easy determination of transformation efficiency in wild type Col-0 plants, and to test for random integration in the double mutants. To be able to efficiently screen gene targeting events, a GFP expression cassette under control of the seed specific 2S promoter (Bensmihen et al., FEBS Letters 561 1-3 (2004): Analysis of an activated ABI5 allele using a new selection method for transgenic Arabidopsis seeds) was inserted into the repair template. The insertion of the repair template into the ADH-1 locus in the Arabidopsis genome results in green fluorescent seeds, which can then easily be identified by fluorescence microscopy.

### Example 3: Stable Transformation of T-DNA by Agrobacteria to Assess Frequency of Random Integration in the Double Mutant Background

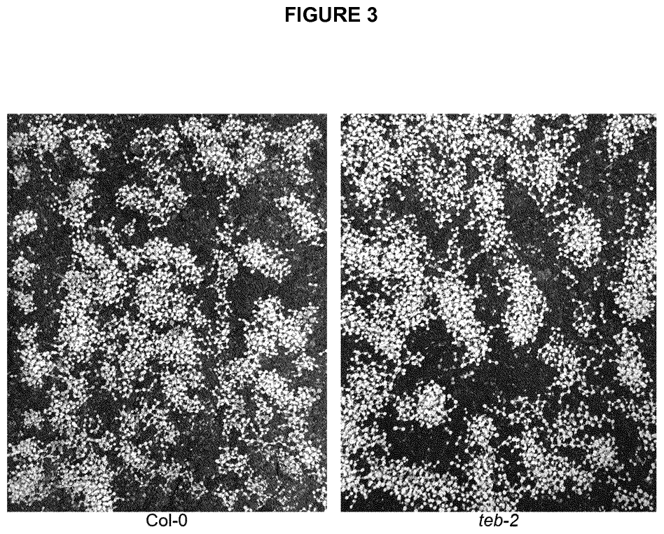

To analyze random integration frequency in the double mutants and the Pol θ single mutants, stable transformation of the gene targeting construct by floral dip Agrobacteria transformation was performed. Since Pol θ mutation was reported to abolish random T-DNA integration into the target genome (van Kregten, M. et al. Nat. Plants 2, 16164 (2016)), it is not possible to determine the rate of transformation by BASTA selection in Pol θ mutant plants. Thus, in order to monitor transformation efficiency wildtype plants were also transformed for each experiment. BASTA selection was then applied to determine transformation efficiency (FIG. 3). Furthermore, a BASTA selection was also done for aliquots of the transformed mutants. The obtained data clearly showed that none of the mutants led to BASTA resistant plants, demonstrating that the random integration of the T-DNA targeting construct was successfully inhibited in single and double Pol θ mutants (FIG. 3).

### Example 4: Agrobacterium tumefaciens Transformation to Assess Gene Targeting Frequency in the Double Mutant Background

To test the gene targeting frequency single and double mutants were transformed with the above described gene targeting construct. First, polQ single mutants were transformed with the gene targeting constructs, following the Arabidopsis floral dip protocol described in Clough et al. (Clough, S. J. and Bent, A. F. (1998) Floral dip: a simplified method for Agrobacterium-mediated transformation of Arabidopsis thaliana. Plant J, 16(6), 735-743). In parallel, wildtype Col-0 plants were transformed to confirm high transformation efficiency. After floral dip transformation, plants were grown for approximately 3 weeks. Then watering was stopped to promote seed maturation and mature seeds were harvested. An aliquot of the seeds was used for BASTA selection, and no BASTA resistant plants were identified in both the teb-2 and the teb-5 polQ mutant plants. In the wildtype plants, a transformation efficiency of ˜1% was confirmed. The results indicate that random integration of T-DNA in the polQ mutant plants is efficiently inhibited.

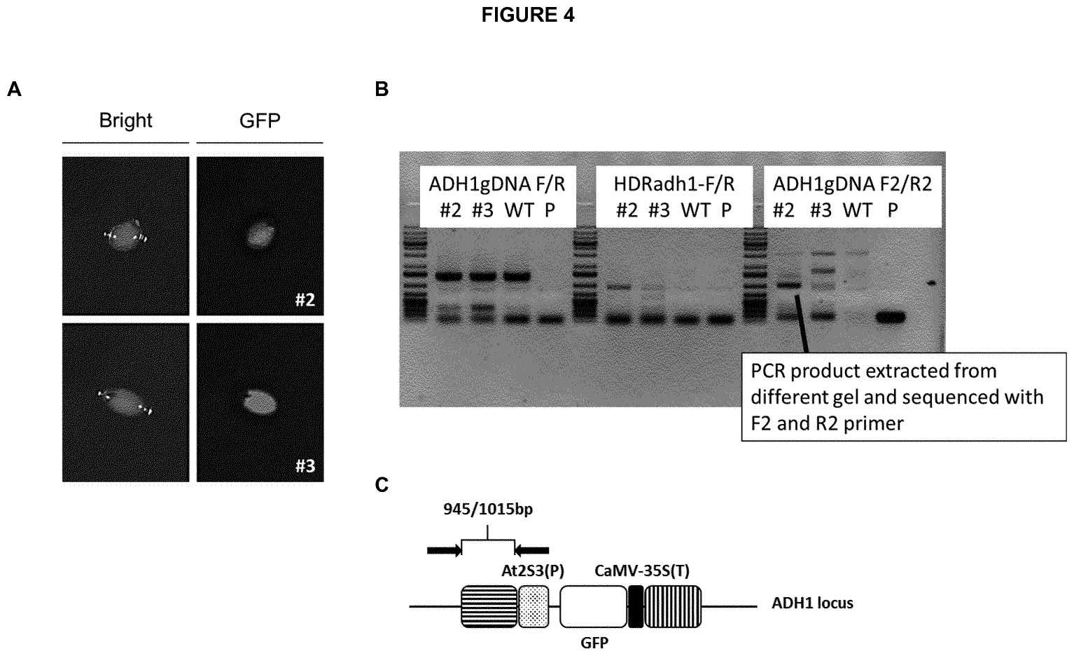

The remaining transformed polQ mutant seeds were then screened for green fluorescent seeds. After three rounds of transformation, only two green fluorescent seeds were indentified, representing an average gene targeting rate of 0.4 HDR events per 100.000 seeds (Table 3). Molecular characterization of these seeds confirmed integration of the repair template into the gene targeting locus of the adh1 gene (FIG. 4).

In the next step, double mutants were transformed with the gene targeting constructs, also following the Arabidopsis floral dip protocol of Clough and Bent (1998). After floral dip transformation, plants were grown for another ˜3 weeks and then watering was stopped to promote seed maturation. Mature seeds were harvested and screened for green fluorescent seeds (Table 3). After three independent transformation experiments, in summary 31 fluorescent seeds were identified in the teb-5×ligIV double mutant, representing an average gene targeting rate rate of 2.9 HDR events per 100.000 seeds (Table 3). Similar results were obtained in the equivalent teb-2×ligIV double mutant, where 13 fluorescent seeds were identified, representing an gene targeting rate of 5.6 HDR events per 100.000 seeds.

The gene targeting rate was also determined in the teb-5×ku70 double mutants. There rounds of transformation experiments were performed as described above. In total, 19 fluorescent seeds were identified in the teb-5×ku70 double mutant, representing an average gene targeting rate of 1.9 HDR events per 100.000 seeds (Table 3).

The obtained data indicate a relative increase in the gene targeting rate in both the polQ-ligIV and polQ-ku70 double mutants compared to the polQ single mutants.

TABLE 3
Summary of transformation experiments, number of total seeds,
fluorescent seeds and the transformation efficiency.

| Floral dip | | No. of | Agrobact. | Number of | Fluorescent | HDR Rate | Transformation | |
|---|---|---|---|---|---|---|---|---|
| exp. No. | Genotype | plants | strain | seeds | seeds | (/100.000) | efficiency | |
| #10 | Col-0 | 48 | AGL1 | 407100 | >>105 | | ~0.8% | (BASTA) |
| | teb-2 | 48 | | 419500 | 0 | 0 | 0% | (BASTA) |
| | teb-5 | 48 | | 447400 | 0 | 0 | 0% | (BASTA) |
| #11 | Col-0 | 48 | GV3101 | 408200 | >>67 | | ~0.5% | (BASTA) |
| | teb-2 | 48 | | 282300 | 0 | 0 | 0% | (BASTA) |
| | teb-5 | 48 | | 315100 | 1 | 0.32 | 0% | (BASTA) |
| #15 | Col-0 | 48 | GV3101 | 269100 | >>6 | | | |
| | teb-2 | 48 | | 257300 | 1 | 0.39 | | |
| | teb-5 | 48 | | 419200 | 0 | 0 | | |
| | teb-5 × ligIV | 48 | | 175600 | 0 | 0 | | |
| | teb-5 × ku70 | 48 | | 113200 | 0 | 0 | | |
| #17 | Col-0 | 108 | GV3101 | 410200 | >>51 | | | |
| | teb-2 × ligIV | 108 | | 233100 | 13 | 5.58 | | |
| | teb-5 × ligIV | 108 | | 200200 | 18 | 8.99 | | |
| | teb-5 × ku70 | 108 | | 233100 | 15 | 6.43 | | |
| #18 | Col-0 | 96 | GV3101 | 913000 | >>13 | | | |
| | teb-5 × ligIV | 96 | | 687400 | 13 | 1.89 | | |
| | teb-5 × ku70 | 96 | | 677700 | 4 | 0.59 | | |

Overall, the herein presented data thus clearly in show dicate that double mutants in Pol θ and Ku70, Ku80 or LigIV result in increased homologous recombination, while the random integration of T-DNA into the plant genome is efficiently inhibited. The herein described methods of the invention therefore provide means to introduce site-specific edits or modifications in a highly precise manner without inserting unwanted mutations or edits into a genome of interest as random/non-predictable integration during repair of an artificially induced double strand break is efficiently inhibited.

### Example 5: Generation of Double Mutants in Arabidposis Thaliana (Arabidopsis)

In addition to the above experiments, further plant models can be provided. To this end, suitable clones are SALK_018851.41.00.x SALK T-DNA homozygous knockout line for At4g32695, SALK_035610.46.30.x SALK T-DNA homozygous knockout line for At4g32700, for KU70: At1g16970; Col-0: SALK_123114 (Heacock et al., 2007), for KU80: At1g48050; Col-0: SAIL_714_A04; Ws: FLAG_396 B06, and for LIG4: At5g57160; Col-0: SALK_044027 (Atlig4-2); Col-0: SAIL_597_D10 (Atlig4-5) (Waterworth et al., 2010), respectively. Crosses can be performed in both direction, with mutant X (Pol θ) as father and mutant Y (Ku70, Ku80 or LigIV) as mother, or vice versa. Crossed plants could then be selfed to fix the mutations in both genes. Progeny of the crosses are then analyzed by specific PCR screening systems for T-DNA integration in both mutated genes, optionally followed by selfing steps. The resulting homozygous double mutants Pol θ//KU70, Pol θ//KU80 and Pol θ//LigIV can be used for all further experiments in Arabidopsis.

During plant growth for described crossing experiments plants and their phenotypes are assessed for potential negative growth impacts.

Further insertion mutant information can be obtained from the SIGnAL website at http://signal.salk.edu. Relevant genetic material suitable for the crosses can be obtained from the SALK T-DNA collection (Alonso, J. M. et al. Genome-wide insertional mutagenesis of Arabidopsis, 2003).

### Example 6: Stable Transformation of T-DNA by Agrobacteria to Assess Frequency of Random Integration in the Double Mutant Background

To further analyze random integration frequency in the double mutants, stable transformation of T-DNA by Agrobacteria transformation is performed. Briefly, Agrobacterium tumefaciens has been transformed with a binary vector containing a nptII resistance gene followed by transformation of Arabidopsis plant material. Any other, or an additional marker, including hygromycin (hyg), sulfadizine or basta, for example, may be used. Arabidopsis plants is then grown to flowering stage at 24° C. day/20° C. night, with 250 μmol photon m⁻² s⁻¹. These plants correspond to the homozygous double mutant lines in Example 1, or non-mutant siblings as controls. To obtain more floral buds per plant, inflorescences can be clipped after most plants have formed primary bolts, relieving apical dominance and encouraging synchronized emergence of multiple secondary bolts. Next, plants are infiltrated or dipped when most secondary inflorescences were about 1-10 cm tall (4-8 days after clipping).

### Example 7: Agrobacterium tumefaciens (Agrobacterium) Transformation

For Agrobacterium transformations, standard protocols, slightly modified in accordance with Clough et al., 1998, The Plant Journal, can be used for the culture of Agrobacterium and the subsequent inoculation of plants. Briefly, Agrobacterium tumefaciens strain AGL1 is used in all experiments. Bacteria are grown to stationary phase in liquid culture at 28° C., 250 r.p.m. in sterilized LB (10 g tryptone, 5 g yeast extract, 5 g NaCl per litre water). Cells are harvested by centrifugation for 20 min at room temperature at about 5,500 g and then resuspended in infiltration medium to a final OD600 of approximately 0.80 prior to use. A revised floral dip inoculation medium may contain 5.0% sucrose and 0.04% Silwet L-77. For floral dip approaches, the inoculum is added to a beaker, plants are dipped into this suspension in an inverted way such that all above-ground tissues are submerged, and plants are then removed after 2-3 min and the procedure is repeated twice. Such dipped plants are removed from the beaker, placed in a plastic tray and covered with a tall clear-plastic dome to maintain humidity. Plants are left in a dark location overnight at 16-18° C. and returned to the light the next day. Plants are grown for a further 3-5 weeks until siliques are brown and dry. Finally, seeds are harvested for further analysis and experiments.

For transient approaches, i.e., when Agrobacterium is used to insert a traditional hairpin DNA construct to be transcribed into a hairpin RNA having RNA silencing capacity, the same Agrobacterium transformation steps as detailed above may be used.

In case that it is intended to transfect a RNAi mediating small RNA directly into a cell, e.g. a (partially) double-stranded RNA, single-stranded sense and/or antisense RNA, a chimeric or synthetic RNA, and/or a chimeric poly-sgRNAgRNA/siRNA to generate a ribo-nucleo particle with a CRISPR nuclease, a direct delivery of the RNA effector, optionally provided in a complex with a site-specific nuclease, e.g., by transfection methods, may be used.

Harvested seeds are, for example, put on hygromycin selection medium. As it is known in the technical field, any other suitable marker, comprising inter alia antibiotic resistance and/or fluorescent markers, may be used, for example Basta or GFP, optionally under the control of tissue-specific and/or inducible or constitutive promoter, e.g. a seed specific 2S promoter (Bensmihen et al., 2014). Notably, fewer or even 0 (zero) transgenic plants would be identified in the transformed double mutants Pol θ//KU70, Pol θ//KU80 or Pol θ//LigIV, respectively. In WT transformation we observed a transformation frequency of about 0.5% after selection. All experiments should be repeated 5 times to ascertain that there is fewer or even no negative selection impact.

### Example 8: Increased Homologous Recombination in Double Mutants (One Circular Vector)

For further testing increased homologous recombination frequency a construct carrying the bar/hyg gene (including a suitable promoter and terminator), flanked by suitable homology regions to the genome (ADH1 locus) may be used. In principle, any target region, gene of interest or even a nucleic acid to be altered of interest, in the genome of a cell of interest may be used. Here the exemplary target locus is the ADH1 locus. Instead of the hyg marker, another selection marker, also including a reporter gene, may be used.

In addition, the vector contains a CRISPR nuclease, including inter alia a Cas or Cpf, CasX or CasY, encoding sequence as effector nuclease and a corresponding sgRNA or crRNA aligning with a region in the target ADH1 locus. WT plants (controls) and double mutants (Pol θ//KU70, Pol θ//KU80, and Pol θ//LigIV, respectively) are transformed by floral dip transformation as described above. T1 seedlings are selected on allyl alcohol and additionally analyzed for stable integration of the bar/hyg gene (or any suitable marker) by qPCR or by other inspections methods depending on the marker gene chosen.

A preferred homologous recombination test may be a fluorescent reporter knock-in to cruciferin such as reported by Shaked et al., 2005, (see, for example, http://www.pnas.org/content/102/34/12265) because the results can be directly measured in the T1 seed. Similar assays with a RFP gene knock-in to a different seed storage gene may be used to obtain optimum marker brightness.

T1 may further analyzed to check if the T-DNA of the binary has been integrated. Depending on whether conventional HR using Agrobacterium in a normal (NHEJ active) environment, or precision HR, as disclosed herein, is used either the full-T-DNA, or only certain regions, or only the nucleic acid sequence of interest will be integrated.

To check if a HR-based repair has occurred, plants can be easily analyzed by PCR and amplicon sequencing based on the available sequence information to demonstrate the improved rate of HR in the identified events in comparison to transformed WT plants. Any increase of HR rate in combination with no random integration will be suitable.

### Example 9: Increased Homologous Recombination in Double Mutants (Two Circular Vectors)

In addition to the above described experiments, increased homologous recombination frequency can be tested by using a construct carrying the bar/hyg gene (including promoter and terminator), flanked by suitable homology regions to the genome (ADH1 locus). In principle, any target region, gene of interest or even a nucleic acid to be altered of interest, in the genome of a cell of interest may be used. Here the exemplary target locus is the ADH1 locus. Instead of the hyg marker, another selection marker, also including a reporter gene, may be used.

In addition, a second vector encoding a Cas or Cpf effector, or any other CRISPR nuclease, as site-specific nuclease and a sgRNA/crRNA aligning with a region in the target ADH1 locus may be used.

WT plants (controls) and double mutants (for example, Pol θ//KU70, Pol θ//KU80, or Pol θ//LigIV, respectively) may be transformed by floral dip transformation as described above. Alternatively, other transformation strategies may be used.

T1 seedlings may be selected on allyl alcohol and additionally analyzed for stable integration of the bar/hyg gene by qPCR. Additionally, T1 can be further analyzed to check if the T-DNA of the binary has been integrated. As a result, it might be found that in none of the selected plants a successful integration of the T-DNA can be detected. To check if a real HR event has occurred, plants can be analyzed by PCR and amplicon sequencing. To check if a HR-based repair has occurred, plants can be easily analyzed by PCR and amplicon sequencing based on the available sequence information to demonstrate the improved rate of HR in the identified events in comparison to transformed WT plants. Any increase of HR rate in combination with no random integration event detected will be suitable.

### Example 10: Increased Homologous Recombination in Protoplasts of Double Mutants (One Circular Vector)

For further testing the effect of the double mutants in different plant material and to demonstrate a broad applicability, increased homologous recombination frequency can be tested using a construct carrying the bar/hyg gene (including suitable promoter and terminator structures), flanked by suitable homology regions to the genome (ADH1 locus) may be used. In principle, any target region, gene of interest or even a nucleic acid to be altered of interest, in the genome of a cell of interest may be used. Here the exemplary target locus is the ADH1 locus. Instead of the hyg marker, another selection marker, also including a reporter gene, may be used.

In addition, a vector containing a CRISPR nuclease and at least one suitable sgRNA or crRNA aligning with a region in the target ADH1 locus is provided. WT protoplasts (controls) and double mutant protoplasts (for example, Pol θ//KU70; Pol θ//KU80, or Pol θ//LigIV, respectively) can be isolated and transformed by polyethylene glycol (PEG) transformation following standard protocols (see, e.g., Methods in Molecular Biology, vol. 82, Arabidopsis Protocols). Protoplasts are analyzed after 48 hr by PCR for stable integration of repair template and/or HR at designated target site. Additionally, HR can be confirmed by sequencing. The frequency is expected to be at least 3-fold higher than the results measured in the transformed WT protoplasts. Any increase of HR rate in combination with no random integration event detected will be suitable.

### Example 11: Increased Homologous Recombination in Protoplasts of Double Mutants (Two Circular Vectors)

For further testing increased homologous recombination frequency, again a construct carrying the bar/hyg gene (including a suitable promoter and terminator), flanked by suitable homology regions to the genome (ADH1 locus) may be used. In principle, any target region, gene of interest or even a nucleic acid to be altered of interest, in the genome of a cell of interest may be used. Here the exemplary target locus is the ADH1 locus. Instead of the hyg marker, another selection marker, also including a reporter gene, may be used. In addition, a second vector containing a CRISPR nuclease encoding sequence as effector nuclease and a corresponding sgRNA/crRNA also comprising a homology region towards the ADH1 locus may be used. Protoplasts of WT plants (controls) and different double mutants (for example, Pol θ//KU70; Pol θ//KU80, or Pol θ//LigIV, respectively) can then be isolated and transformed by PEG transformation following standard protocols. Protoplasts are analyzed after 48 hr by PCR for stable integration of repair template and/or HR at designated target site. Additionally, HR can be confirmed by sequencing. For this set-up in the protoplasts, the frequency is expected to be at least 3-fold higher than the results measured in the transformed WT protoplasts. Any increase of HR rate in combination with no random integration event detected will be suitable.

### Example 12: Increased Homologous Recombination in Protoplasts of Double Mutants (One Linearized Vector)

As a further experiment in the protoplast test series, increased homologous recombination frequency can be tested using a linearized vector. Again, a construct carrying the bar/hyg gene (including a suitable promoter and terminator), flanked by suitable homology regions to the genome (ADH1 locus) may be used. In principle, any target region, gene of interest or even a nucleic acid to be altered of interest, in the genome of a cell of interest may be used. Here the exemplary target locus is the ADH1 locus. Instead of the hyg marker, another selection marker, also including a reporter gene, may be used. In addition, a second vector containing a CRISPR nuclease of interest and sgRNA/crRNA as detailed above may be used. Both vectors can be linearized by a unique restriction enzyme, for example NotI, AscI, or another, preferably 8 base, cutter. Protoplasts of WT plants (controls) and double mutants (for example, Pol θ//KU70; Pol θ//KU80, or Pol θ//LigIV, respectively) may be isolated and transformed by PEG transformation as described above. Protoplasts were then analyzed after 48 hr by PCR for stable integration of repair template and/or HR at designated target site. Additionally, HR can be confirmed by sequencing. For this set-up, the frequency is expected to be at least 1.25 to 1.5-fold higher than the results measured in the transformed WT protoplasts. Any increase of HR rate in combination with no random integration event detected will be suitable.

### Example 13: Triple and Quadruple Mutants

Based on the material detailed in Example 1 above, triple and quadruple mutants may be constructed in the Arabidopsis background to expand the toolkit available for optimizing highly site-specific genome editing experiments in plant cells. By conventional crossing and breeding, for example, a Pol θ//KU70//KU80 (P78), Pol θ//KU80//LigIV (P8L), a Pol θ//KU70//LigIV (P7L), and a Pol θ//KU70//KU80//LigIV (P78L) mutant can thus be created.

Initial tests, again using both Agrobacterium and protoplast transformation/transfection using either one or more vectors, optionally linearized for protoplast transfections, of a bar/hyg construct together with a CRISPR nuclease as site-specific effector nuclease can then revealed that certain mutants, for example, P7L or P8L, or even more dominantly the P78L mutant might have even better results in enhancing the transformation efficiency during GE in comparison to the double mutants.

### Example 14: Transient Approach—RNAi

Transient plant transformation is becoming of increasing importance. For testing increased homologous recombination frequency in a transient set-up, again a construct carrying the bar/hyg gene (including a suitable promoter and terminator), flanked by suitable homology regions to the genome (ADH1 locus) may be used. In principle, any target region, gene of interest or even a nucleic acid to be altered of interest, in the genome of a cell of interest may be used. Here the exemplary target locus is the ADH1 locus. Instead of the hyg marker, another selection marker, also including a reporter gene, may be used. In addition, the vector can contain a CRISPR nuclease site-specific effector coding sequence and the cognate sgRNA/crRNA also against a region in the ADH1 locus as described above.

A second vector may be used carrying a traditional hairpin DNA expression cassette against Pol θ and KU70, or KU80, or LigIV, or any other combination as detailed for the double, triple and quadruple mutants detailed above. As an alternative, the interfering RNA can be delivered as double-stranded RNA, as single-stranded antisense RNA, or as chimeric poly-sgRNA/siRNA sequences which generate multiple sgRNA-CRISRPR nuclease RNP complexes upon the Dicer-mediated digestion of the siRNA parts, leading to more efficient disruption of the target gene in cells (Ha J. S. et al., Journal of Controlled Release 250 (2017) 27-35). HR can be analyzed by PCR and amplicon sequencing.

Notably, the transient down-regulation of Pol θ and a further player involved in NHEJ is of particular interest in the context of targeted GE, as there might be no interest in propagating a knock-out for Pol θ, KU70, KU80, and/or LigIV stably inherited to a progenitor cell, but it might rather be of interest to perform the down-regulation of Pol θ, KU70, KU80, and/or LigIV just before a targeted GE of a nucleic acid, a gene, or a locus of interest is performed to maintain the integrity of the endogenous NHEJ pathway in progeny cells and plants.

### Example 15: Transient Approach—Protein Interference

To further test whether increased homologous recombination frequency can be obtained in a transient knock-down system, again a construct carrying the bar/hyg gene (including a suitable promoter and terminator), flanked by suitable homology regions to the genome (ADH1 locus) may be used. In principle, any target region, gene of interest or even a nucleic acid to be altered of interest, in the genome of a cell of interest may be used. Here the exemplary target locus is the ADH1 locus. Instead of the hyg marker, another selection marker, also including a reporter gene, may be used. In addition, the vector can contain a CRISPR nuclease site-specific effector coding sequence and the cognate sgRNA/crRNA also against a region in the ADH1 locus as described above.

Protein interference with these enzymes can be induced by delivering of adenovirus 4 E1B55K and E4orf6 proteins according to SEQ ID NO: 79 and 81 which specifically inhibit LigIV by delivering small chemical inhibitors of these enzymes such as, for example, SCR7, W7, Vanillin, NU7026, NU7441 (PLOS ONE 11(9): e0163049) which inhibits LigIV, DNA protein kinases, Ku cofactor synthesis; or by any combination. Again, this attempt is particularly suitable for plant genome engineering, where a permanent knock-out of LigIV, KU70, KU80 and/or Pol θ might not be envisaged. HR efficiency and frequency can be analyzed by PCR and amplicon sequencing.

### Example 16: Using NHEJ Interference with GE in Zea mays

Zea mays (or corn, maize) represents a major crop plant worldwide. To transfer the findings of the above examples from the dicot model organism to the monocot maize as relevant crop plant for GE, the experiments done in Arabidopsis can also transferred to the maize model.

The Maize GDB was used to search by sequence for suitable mutant seed stocks. Iterative BLAST analyses were performed in parallel for the relevant genes of interest encoding maize LigIV, KU70, KU80 and/or Pol θ. The insertion of a MU transposon 70 bp upstream of the ATG in the 5′UTR was identified for maize gene GRMZM2G151944. Maize seeds can then be searched on http://teosinte.uoregon.edu/mu-illumina/ from the University of Oregon providing access to a subset of the Mu insertions detected by Mu-Illumine (see https://www.ncbi.nlm.nih.gov/pubmed/20409008) sequencing during mutant cloning efforts involving the Photosynthesis Mutant Library (see http://pml.uoregon.edu/photosyntheticml.html). The posted insertions map between 150 bp upstream of the annotated start codon and 150 bp downstream of the annotated stop codon of gene models in the Filtered Gene Set from Maize Genome Assembly AGPv3 (www.gramene.org). Insertions that map more distant to genes rarely disrupt gene expression; due to limited resources, so that these are not made available.

Due to homologies to a relevant rice DNA polymerase (Os12g19370.1), GRMZM2G151944 containing maize seeds can be suitable.

For KU70, a seed stock insertion site alignment for a known KU70 sequence showed an insertion at the very end of the KU70 gene of maize. The relevant seeds can be ordered at http://teosinte.uoregon.edu/mu-illumina/?maize=GRMZM2G414496#.

For KU80, stocks of uniform MU insertions in the KU80 gene were identified to be Mu1089096, 1043955, 1089097, 1058684 (https://www.maizegdb.org) and the respective seeds can be ordered.

For maize DNA ligase IV (LigIV) uniform MU insertion seed stocks are Mu1009698::Mu Stocks:uFMu-00167; Mu1089771::Mu stocks:uFMu-11366 and mu1044651::mu stocks:UFMu-05547.

First, the available single mutants can be checked for growth performance and impact of mutations on development. In parallel it can be tested, if the mutants are indeed mutated at the desired positions by PCR. To this end, a qPCR system can be established to suitably measure the transcription of the individual genes and the transcription was measured in cDNA

If mutants are confirmed mutants can be used for further experiments. Otherwise different strategies to generate the mutants are possible, like TILLING, GE, GE-base-editors, and the like.

The term “TILLING” or “Targeting Induced Local Lesions in Genomes” describes a well-known reverse genetics technique designed to detect unknown SNPs (single nucleotide polymorphisms) in genes of interest which is widely employed in plant and animal genomics. The technique allows for the high-throughput identification of an allelic series of mutants with a range of modified functions for a particular gene. TILLING combines mutagenesis (e.g., chemical or via UV-light) with a sensitive DNA screening-technique that identifies single base mutations.

Meanwhile, as it is known to the skilled person, TILLING has been extended to many plant species and becomes of paramount importance to reverse genetics in crops species. A major recent change to TILLING has been the application of next-generation sequencing (NGS) to the process, which permits multiplexing of gene targets and genomes.. Because it is readily applicable to most plants, it remains a dominant non-transgenic method for obtaining mutations in known genes and thus represents a readily available method for non-transgenic approaches according to the methods of the present invention. As it is known to the skilled person, TILLING usually comprises the chemical mutagenesis, e.g., using ethyl methanesulfonate (EMS), or UV light induced modification of a genome of interest, together with a sensitive DNA screening-technique that identifies single base mutations in a target gene.

Generally, analysis of increased HR by applying CRISPR nucleases and repair templates in maize may use different variants (single vector, multiple vector, circular, linear, etc.) for the different mutant combinations. T1 seedlings need to be analyzed for HR and for potential stable integration of the T-DNA.

Furthermore, nptII based selection and PMI based selection, or bar based selection may be used. In terms of loci for doing integration assays CDS fusion insertion into highly expressed genes like Alpha Tubulin (GRMZM2G152466), Aconitate hydratase (GRMZM2G020801), or HSP70 may be suitable for better selection.

1. A method for modifying the genetic material of a cellular system at a predetermined location with at least one nucleic acid sequence of interest, wherein the method comprises the following steps:
(a) providing a cellular system comprising a Polymerase theta enzyme, or a sequence encoding the same, and one or more further enzymes of a NHEJ pathway, or one or more sequences encoding the same;
(b) inactivating or partially inactivating the Polymerase theta enzyme, or the sequence encoding the same, and inactivating or partially inactivating one or more further DNA repair enzymes of a NHEJ pathway, or one or more sequences encoding the same;
(c) introducing into the cellular system
(i) the at least one nucleic acid sequence of interest, optionally flanked by one or more homology sequences complementary to one or more nucleic acid sequences adjacent to the predetermined location, and
(ii) at least one site-specific nuclease, or a sequence encoding the same, the site-specific nuclease inducing a double-strand break at the predetermined location;
(d) optionally: determining the presence of the modification at the predetermined location in the genetic material of the cellular system; and
(e) obtaining a cellular system comprising a modification at the predetermined location of the genetic material of the cellular system.

2. The method of claim 1, wherein the method comprises the additional step:
(f) restoring an activity of the inactivated or partially inactivated Polymerase theta enzyme and/or restoring an activity of the one or more further inactivated or partially inactivated DNA repair enzymes of a NHEJ pathway in the cellular system comprising a modification at the predetermined location, or in a progeny system thereof.

3. The method according to claim 1, wherein the Polymerase theta to be inactivated or partially inactivated comprises an amino acid sequence according to SEQ ID NO: 2, 7, 8, 9 or 10, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 2, 7, 8, 9 or 10, respectively; or is encoded by a nucleic acid sequence according to SEQ ID NO: 1, 3, 4, 5 or 6, or a nucleic acid having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID No: 1, 3, 4, 5 or 6, respectively.

4. The method according to claim 1, wherein the one or more further DNA repair enzymes of a NHEJ pathway to be inactivated or partially inactivated are independently selected from the group consisting of Ku70, Ku80, DNA-dependent protein kinase, Ataxia telangiectasia mutated (ATM), ATM—and Rad3—related (ATR), Artemis, XRCC4, DNA ligase IV and XLF, or any combination thereof.

5. The method according to claim 4, wherein at least two, at least three, or at least four further DNA repair enzymes of a NHEJ pathway are inactivated or partially inactivated, preferably wherein at least Ku70 and DNA ligase IV, or wherein at least Ku80 and DNA ligase IV are inactivated or partially inactivated.

6. The method according to claim 1, wherein the one or more further DNA repair enzymes of a NHEJ pathway to be inactivated or partially inactivated is Ku70, or a nucleic acid sequence encoding the same, wherein the Ku70 comprises an amino acid sequence according to SEQ ID NO: 12, 18, 19 or 20, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 12, 18, 19 or 20, respectively, or wherein the nucleic acid sequence encoding the same comprises a sequence according to SEQ ID NO: 11, 13, 14, 15, 16 or 17, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 11, 13, 14, 15, 16 or 17, respectively, and/or wherein the one or more further DNA repair enzymes of a NHEJ pathway to be inactivated or partially inactivated is Ku80, or a nucleic acid sequence encoding the same, wherein the Ku80 comprises an amino acid sequence according to SEQ ID NO: 22, 23, 24 or 29, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 22, 23, 24 or 29, respectively, or wherein the nucleic acid sequence encoding the same comprises a sequence according to SEQ ID NO: 21, 25, 26, 27 or 28, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 21, 25, 26, 27 or 28, respectively, and/or wherein the one or more further DNA repair enzymes of a NHEJ pathway to be inactivated or partially inactivated is DNA-dependent protein kinase, or a nucleic acid sequence encoding the same, wherein the DNA-dependent protein kinase comprises an amino acid sequence according to SEQ ID NO: 32, 33 or 35, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 32, 33 or 35, respectively, or wherein the nucleic acid sequence encoding the same comprises a sequence according to SEQ ID NO: 30, 31 or 34, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 30, 31 or 34, respectively, and/or wherein the one or more further DNA repair enzymes of a NHEJ pathway to be inactivated or partially inactivated is ATM, or a nucleic acid sequence encoding the same, wherein the ATM comprises an amino acid sequence according to SEQ ID NO: 37, 38, 39, 41, 42, 43, 44, 45, 46, 47 or 48, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 37, 38, 39, 41, 42, 43, 44, 45, 46, 47 or 48, respectively, or wherein the nucleic acid sequence encoding the same comprises a sequence according to SEQ ID NO: 36 or 40, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 36 or 40, respectively, and/or wherein the one or more further DNA repair enzymes of a NHEJ pathway to be inactivated or partially inactivated is ATM—and Rad3—related (ATR), or a nucleic acid sequence encoding the same, wherein the ATR comprises an amino acid sequence according to SEQ ID NO: 50, 51, 52, 53, 55 or 56, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 50, 51, 52, 53, 55 or 56, respectively, or wherein the nucleic acid sequence encoding the same comprises a sequence according to SEQ ID NO: 49 or 54, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO:49 or 54, respectively, and/or wherein the one or more further DNA repair enzymes of a NHEJ pathway to be inactivated or partially inactivated is Artemis, or a nucleic acid sequence encoding the same, wherein the Artemis comprises an amino acid sequence according to SEQ ID NO: 60, 61, 62 or 64, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 60, 61, 62 or 64, respectively, or wherein the nucleic acid sequence encoding the same comprises a sequence according to SEQ ID NO: 57, 58, 59 or 63, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 57, 58, 59 or 63, respectively, and/or wherein the one or more further DNA repair enzymes of a NHEJ pathway to be inactivated or partially inactivated is XRCC4, or a nucleic acid sequence encoding the same, wherein the XRCC4 comprises an amino acid sequence according to SEQ ID NO: 66, 67 or 69, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 66, 67 or 69, respectively, or wherein the nucleic acid sequence encoding the same comprises a sequence according to SEQ ID NO: 65 or 68, or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 65 or 68, respectively, and/or wherein the one or more further DNA repair enzymes of a NHEJ pathway to be inactivated or partially inactivated is DNA ligase IV, or a nucleic acid sequence encoding the same, wherein the DNA ligase IV comprises an amino acid sequence according to SEQ ID NO: 71, 72, 76 or 77, or an amino acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 71, 72, 76 or 77, respectively, or wherein the nucleic acid sequence encoding the same comprises a sequence according to SEQ ID NO: 70, 73, 74 or 75 or a nucleic acid sequence having at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the sequence set forth in SEQ ID NO: 70, 73, 74 or 75, respectively, and/or wherein the one or more further DNA repair enzymes of a NHEJ pathway to be inactivated or partially inactivated is XLF, or a nucleic acid sequence encoding the same.

7. The method according to claim 1, wherein the at least one nucleic acid sequence of interest is provided as part of at least one vector, or as at least one linear molecule.

8. The method according to claim 7, wherein the at least one vector is introduced into the cellular system by biological or physical means, including transfection, transformation, including transformation by Agrobacterium spp., preferably by Agrobacterium tumefaciens, a viral vector, biolistic bombardment, transfection using chemical agents, including polyethylene glycol transfection, or any combination thereof.

9. The method according to claim 1, wherein the at least one site-specific nuclease, or the sequence encoding the same, is introduced into the cellular system by biological or physical means, including transfection, transformation, including transformation by Agrobacterium spp., preferably by Agrobacterium tumefaciens, a viral vector, bombardment, transfection using chemical agents, including polyethylene glycol transfection, or any combination thereof.

10. The method according to claim 1, wherein the at least one site-specific nuclease or a catalytically active fragment thereof, is introduced into the cellular system as a nucleic acid sequence encoding the site-specific nuclease or the catalytically active fragment thereof, wherein the nucleic acid sequence is part of at least one vector, or wherein the at least one site-specific nuclease or the catalytically active fragment thereof, is introduced into the cellular system as at least one amino acid sequence.

11. The method according to claim 1, wherein the at least one nucleic acid sequence of interest to be introduced into a cellular system is selected from the group consisting of: a transgene, a modified endogenous gene, a synthetic sequence, an intronic sequence, a coding sequence or a regulatory sequence.

12. The method according to claim 1, wherein the at least one nucleic acid sequence of interest to be introduced into a cellular system is a transgene, wherein the transgene comprises a nucleic acid sequence encoding a gene of a genome of an organism of interest, or at least a part of said gene.

13. The method according to claim 1, wherein the at least one nucleic acid sequence of interest to be introduced into a cellular system at a predetermined location is a transgene of an organism of interest, wherein the transgene or part of the transgene is selected from the group consisting of a gene encoding resistance or tolerance to abiotic stress, including drought stress, osmotic stress, heat stress, cold stress, oxidative stress, heavy metal stress, nitrogen deficiency, phosphate deficiency, salt stress or waterlogging, herbicide resistance, including resistance to glyphosate, glufosinate/phosphinotricin, hygromycin, protoporphyrinogen oxidase (PPO) inhibitors, ALS inhibitors, and Dicamba, a gene encoding resistance or tolerance to biotic stress, including a viral resistance gene, a fungal resistance gene, a bacterial resistance gene, an insect resistance gene, or a gene encoding a yield related trait, including lodging resistance, flowering time, shattering resistance, seed color, endosperm composition, or nutritional content.

14. The method according to claim 1, wherein the at least one nucleic acid sequence of interest to be introduced into a cellular system at a predetermined location is at least part of a modified endogenous gene of an organism of interest, wherein the modified endogenous gene comprises at least one deletion, insertion and/or substitution of at least one nucleotide in comparison to the nucleic acid sequence of the unmodified endogenous gene.

15. The method according to claim 1, wherein the at least one nucleic acid sequence of interest to be introduced into a cellular system at a predetermined location is at least part of a modified endogenous gene of an organism of interest, wherein the modified endogenous gene comprises at least one of a truncation, duplication, substitution and/or deletion of at least one nucleic acid position encoding a domain of the modified endogenous gene.

16. The method according to claim 1, wherein the at least one nucleic acid sequence of interest to be introduced into a cellular system at a predetermined location is at least part of a regulatory sequence, wherein the regulatory sequence comprises at least one of a core promoter sequence, a proximal promoter sequence, a cis regulatory sequence, a trans regulatory sequence, a locus control sequences, an insulator sequence, a silencer sequence, an enhancer sequence, a terminator sequence, and/or any combination thereof.

17. The method according to claim 1, wherein the at least one site-specific nuclease comprises a zinc-finger nuclease, a transcription activator-like effector nuclease, a CRISPR/Cas system, including a CRISPR/Cas9 system, a CRISPR/Cpf1 system, a CRISPR/CasX system, a CRISPR/CasY system, an engineered homing endonuclease, and a meganuclease, and/or any combination, variant, or catalytically active fragment thereof

18. The method according to claim 1. wherein the one or more nucleic acid sequences flanking the at least one nucleic acid sequence of interest at the predetermined location is/are at least 85%-100% complementary to the one or more nucleic acid sequences) sequences adjacent to the predetermined location, upstream and/or downstream from the predetermined location, over the entire length of a respective adjacent region.

19. The method according to claim 1, wherein the genetic material of the cellular system is selected from the group consisting of a protoplast, a viral genome transferred in a recombinant host cell, a eukaryotic or prokaryotic cell, tissue, or organ, and a eukaryotic or prokaryotic organism.

20. The method according to claim 19, wherein the eukaryotic cell is a plant cell, or an animal cell.

21. The method according to claim 19, wherein the eukaryotic organism is a plant, or a part of a plant.

22. The method according to claim 21, wherein the part of the plant is selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

23. The method according to claim 1, wherein the genetic material of the cellular system is, or originates from, a plant species selected from the group consisting of: Hordeum vulgare, Hordeum bulbusom, Sorghum bicolor, Saccharum officinarium, Zea mays, Setaria italica, Oryza minula, Oriza sativa, Oryza australiensis, Oryza alta, Triticum aestivtun, Secale cereale, Malus domestica, Brachypodium distachyon, Hordeum marinum, Aegilops tauschii, Danciis glochidialus, Beta vulgaris, Daucus pusillus, Daucus muricatus, Daucus carota, Eucalyptus grandis, Nicotiana sylvestris, Nicotiana tomentosiformis, Nicotiana tabacum, Solatium lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. saliva. Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris. Glycine max. Astragalus sinicus, Lotus japonicas, Torenia fournieri, Allium cepa, Allium fistulosum, Allium sativum, and Album tuberosum.

24. A cellular system obtained by the method according to claim 1.

25. A cellular system comprising an inactivated or partially inactivated Polymerase theta (Pol theta) enzyme and one or more further inactivated or partially inactivated DNA repair enzymes of a NHEJ pathway,
wherein the modified cellular system is selected from the group consisting of one or more plant cells, a plant, and parts of a plant.

26. The cellular system according to claim 25, wherein the one or more parts of the plant are selected from the group consisting of leaves, stems, roots, emerged radicles, flowers, flower parts, petals, fruits, pollen, pollen tubes, anther filaments, ovules, embryo sacs, egg cells, ovaries, zygotes, embryos, zygotic embryos, somatic embryos, apical meristems, vascular bundles, pericycles, seeds, roots, and cuttings.

27. The cellular system according to claim 25, wherein the one or more plant cells, the plant or the parts of a plant originate From a plant species selected from the group consisting of: Hordeum vulgare, Hordeum bulbusom, Sorghum bicolor, Saccharum officinarium, Zea mays, Setaria italica, Oryza minuta, Oriza sativa, Oryza auslraliensis, Oryza alata, Triticum aestivum, Secale cereale, Malus domestica, Brachypodium distachyon, Hordeum marinum, Aegilops lauschii, Daucus glochidiatus, Beta vulgaris, Daucus pusillus, Daucus muricatus, Daucus carota, Eucalyptus graudis, Nicotiana sylvestris, Nicotiana tomentosiformis, Nicotiana labacum, Solatium lycopersicum, Solanum tuberosum, Coffea canephora, Vitis vinifera, Erythrante guttata, Genlisea aurea, Cucumis sativus. Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oeleracia, Brassica rapa, Raphanus sativus, Brassica juncea, Brassica nigra, Eruca vesicaria subsp. sativa. Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides. Phaseolus vulgaris. Glycine max. Astragalus sinicus, Lotus japonicas, Torenia fournieri, Allium cepa, Allium fistulosum, Allium sativum, and Allium tuberosum.
